SEQUENCE LISTING (23 sequences)

SEQ ID NO: 1 - protein, 36 aa, Pereskia bleo
QCKPNGAKCTEISIPPCCSNFCLRYAGQKSGTCANR

SEQ ID NO: 2 - protein, 32 aa, Artificial
  C-rich motif
  MISC_FEATURE (2)..(7): X can be any amino acid with the exception of C
  MISC_FEATURE (9)..(15): X can be any amino acid with the exception of C
  MISC_FEATURE (18)..(20): X can be any amino acid with the exception of C
  MISC_FEATURE (22)..(31): X can be any amino acid with the exception of C
CXXXXXXCXXXXXXXCCXXXCXXXXXXXXXXC

SEQ ID NO: 3 - protein, 5 aa, Artificial
  Loop 4 motif
  MISC_FEATURE (2)..(2): X can be any amino acid with the exception of C
  MISC_FEATURE (4)..(4): X can be any amino acid with the exception of C
  MISC_FEATURE (5)..(5): X is R or K
YXGXX

SEQ ID NO: 4 - protein, 32 aa, Artificial
  C-rich peptide motif
  MISC_FEATURE (2)..(7): X can be any amino acid with the exception of C
  MISC_FEATURE (9)..(15): X can be any amino acid with the exception of C
  MISC_FEATURE (18)..(20): X can be any amino acid with the exception of C
  MISC_FEATURE (22)..(23): X can be any amino acid with the exception of C
  MISC_FEATURE (25)..(25): X can be any amino acid with the exception of C
  MISC_FEATURE (27)..(27): X can be any amino acid with the exception of C
  MISC_FEATURE (28)..(28): X is K or R
  MISC_FEATURE (29)..(31): X can be any amino acid with the exception of C
CXXXXXXCXXXXXXXCCXXXCXXYXGXXXXXC

SEQ ID NO: 5 - protein, 35 aa, Artificial
  C-rich peptide motif
  MISC_FEATURE (2)..(2): X is K or R
  MISC_FEATURE (4)..(4): X is any amino acid with the exception of C
  MISC_FEATURE (6)..(6): X is any amino acid with the exception of C
  MISC_FEATURE (7)..(7): X is K or R
  MISC_FEATURE (9)..(13): X is any amino acid with the exception of C
  MISC_FEATURE (18)..(20): X is any amino acid with the exception of C
  MISC_FEATURE (22)..(22): X is any amino acid with the exception of C
  MISC_FEATURE (23)..(23): X is K or R
  MISC_FEATURE (25)..(25): X is any amino acid with the exception of C
  MISC_FEATURE (27)..(27): X is any amino acid with the exception of C
  MISC_FEATURE (28)..(28): X is K or R
  MISC_FEATURE (29)..(29): X is any amino acid with the exception of C
  MISC_FEATURE (31)..(31): X is any amino acid with the exception of C
  MISC_FEATURE (33)..(34): X is any amino acid with the exception of C
  MISC_FEATURE (35)..(35): X is K or R
CXPXGXXCXXXXXPPCCXXXCXXYXGXXXGXCXXX

SEQ ID NO: 6 - protein, 35 aa, Artificial
  C-rich peptide motif
  MISC_FEATURE (4)..(4): X can be any amino acid with the exception of C
  MISC_FEATURE (6)..(6): X can be any amino acid with the exception of C
  MISC_FEATURE (9)..(9): X can be any amino acid with the exception of C
  MISC_FEATURE (11)..(13): X can be any amino acid with the exception of C
  MISC_FEATURE (18)..(20): X can be any amino acid with the exception of C
  MISC_FEATURE (22)..(22): X can be any amino acid with the exception of C
  MISC_FEATURE (25)..(25): X can be any amino acid with the exception of C
  MISC_FEATURE (27)..(27): X can be any amino acid with the exception of C
  MISC_FEATURE (29)..(29): X can be any amino acid with the exception of C
  MISC_FEATURE (31)..(31): X can be any amino acid with the exception of C
  MISC_FEATURE (33)..(34): X can be any amino acid with the exception of C
CKPXGXKCXEXXXPPCCXXXCXRYXGXKXGXCXXR

SEQ ID NO: 7 - protein, 36 aa, Artificial
  Bleogen pB1 variant
  MISC_FEATURE (25)..(25): X is D-tyrosine
QCKPNGAKCTEISIPPCCSNFCLRXAGQKSGTCANR

SEQ ID NO: 8 - protein, 36 aa, Artificial
  Bleogen pB1 variant
  MISC_FEATURE (29)..(29): X is D-lysine
QCKPNGAKCTEISIPPCCSNFCLRYAGQXSGTCANR

SEQ ID NO: 9 - protein, 36 aa, Artificial
  Bleogen pB1 variant
  MISC_FEATURE (25)..(25): X is D-tyrosine
  MISC_FEATURE (29)..(29): X is D-lysine
QCKPNGAKCTEISIPPCCSNFCLRXAGQXSGTCANR

SEQ ID NO: 10 - protein, 53 aa, Homo sapiens
NSDSECPLSHDGYCLHDGVCMYIEALDKYACNCVVGYIGERCQYRDLKWWELR

SEQ ID NO: 11 - protein, 33 aa, Achyranthes bidentata
NCESGTSCIPGAQHNCCSGVCVPIVTIFYGVCY

SEQ ID NO: 12 - protein, 36 aa, Salix viminalis
QCKPNGARCTESSIPPCCSRFCLRYPGQRWGRCANR

SEQ ID NO: 13 - protein, 36 aa, Salix viminalis
QCKPNGAKCTEISIPPCCSGYCLRYAGQKSGTCTNR

SEQ ID NO: 14 - protein, 33 aa, Cimicifuga racemose
QCKPNGAKCTEISIPPCCSGYCLRYAGQKSGTC

SEQ ID NO: 15 - protein, 5 aa, Artificial
  Peptide motif
  MISC_FEATURE (4)..(4): X may be any amino acid with the exception of C
YAGXK

SEQ ID NO: 16 - protein, 5 aa, Artificial
  Peptide motif
  MISC_FEATURE (2)..(2): X may be any amino acid with the exception of C
YXGQK

SEQ ID NO: 17 - protein, 34 aa, Hibiscus sabdariffa
CVSSGIVDACSECCEPDKCIIMLPTWPPRYVCSV

SEQ ID NO: 18 - DNA, 22 bp, Artificial
  Primer
gcctctcttactaccactcacc

SEQ ID NO: 19 - DNA, 22 bp, Artificial
  Primer
agatggcagtgaccgtgggaat

SEQ ID NO: 20 - DNA, 23 bp, Artificial
  Primer
ccttgaaagctcagaactcggag

SEQ ID NO: 21 - DNA, 22 bp, Artificial
  Primer
tgctgcgttagcatgagttggc

SEQ ID NO: 22 - DNA, 22 bp, Artificial
  Primer
gtctcctctgacttcaacagcg

SEQ ID NO: 23 - DNA, 22 bp, Artificial
  Primer
accaccctgttgctgtagccaa

(19) United States
(12) Patent Application Publication
(10) Pub. No.: US 2022/0257706 A1
(43) Pub. Date: Aug. 18, 2022

(54) EPIDERMAL GROWTH FACTOR RECEPTOR (EGFR) LIGANDS

(71) Applicant: NANYANG TECHNOLOGICAL UNIVERSITY, Singapore (SG)

(72) Inventors: James P. TAM, Singapore (SG); Shining LOO, Singapore (SG); Antony KAM, Singapore (SG)

(21) Appl. No.: 17/625,314

(22) PCT Filed: Jul. 9, 2020
(86) PCT No.: PCT/SG2020/050392

§ 371 Date: Jan. 6, 2022

(30) Foreign Application Priority Data
Jul. 10, 2019 (SG) 10201906403T

(51) Int. Cl. A61K 38/16; A61K 8/64; A61P 17/02; A61K 47/69; A61K 9/00

(52) U.S. Cl. CPC A61K 38/168; A61K 8/64; A61K 9/0014; A61K 47/6953; A61P 17/02

(57) ABSTRACT

The present invention relates to hyper-stable EGFR peptide ligands that are plant-derived, particularly isolated from Pereskia belo, as well as variants thereof. Also encompassed are nucleic acids encoding them, host cells comprising said nucleic acid, composition comprising peptide ligands, methods and therapeutic uses thereof.

### CROSS-REFERENCE TO RELATED APPLICATIONS

This application claims the benefit of priority of Singapore Patent Application No. 10201906403T filed Jul. 10, 2019, the contents of which being hereby incorporated by reference in its entirety for all purposes.

### FIELD OF THE INVENTION

The present invention lies in the technical field of peptide/protein technology and specifically relates to ligands for EGF receptor that have improved stability and methods and uses thereof.

### BACKGROUND OF THE INVENTION

Chronic wounds and attendant consequences of severe pain, amputation, and disability, remain an important health concern and increasing socio-economic burden. At least 15% of diabetic patients will develop chronic ulcers on their feet. The total medical cost for managing this ailment was estimated US$9-13 billion in the United States. In this aspect, growth factors which play important roles in proliferation, migration, and differentiation, offer promise as a therapeutic intervention for wound management. Epidermal growth factor (EGF), the first growth factor discovered in 1960, has been clinically approved in several countries for treating diabetic foot ulcers. However, EGF displays poor proteolytic stability in the microenvironment of chronic wounds and frequent applications are required to achieve the desired therapeutic effects. Recent findings suggest that topical application of EGF combined with protease inhibitors could improve wound healing compared to EGF alone.

Epidermal growth factor receptor (EGFR/ErbB1/HER) is a primary tyrosine kinase receptor (RTK) that initiates diverse cellular responses required for developmental growth. Epidermal growth factor (EGF) was the first EGFR agonist discovered. Since then, EGFR agonists have been found in nearly all forms of organisms, such as mammals, viruses, insects, and nematodes, but not in plants. They include seven related mammalian EGFR agonists, ranging from 53 to 132 amino acid residues, three of which are high-affinity agonists: EGF, transforming growth factor-alpha (TGF-α), and heparin-binding epidermal growth factor (hb-EGF). The remaining four, betacellulin, amphiregulin, epiregulin, and epigen, are low-affinity agonists. All possess the consensus EGF-like domain. Thus far, there is no exception of an EGFR agonist without a canonical EGF domain. These agonists produce dissimilar biological responses due to their complex interactions with different members of the Erb family of RTKs. EGFR ligands can also be found in non-mammalian sources, such as Poxviruses (Vaccina growth factor, Myxoma virus growth factor and Shope fibroma virus growth factor), C. elegans (lin-3), and Drosophila (spitz).

EGFR ligands play an important role in cell proliferation, survival, and differentiation through ligand-dependent EGFR activation. All known EGFR peptide agonists share an evolutionarily conserved EGF-like domain, consisting of a cysteine motif, disulfide connectivity (Cys I-III, Cys II-IV, Cys V-VI), and three-loop structure with a double-stranded anti-parallel β-sheet. EGF was introduced as a regenerative medicine in clinical settings, however, its applications were limited by its low in vivo stability.

To date, EGF is a major active ingredient in a number of pharmaceuticals for treating diabetic foot ulcers and in cosmetics used to help rejuvenation of skin, with an estimated market size of US$102 M in 2016 and an average annual growth rate of 6.27% in the skin care industry. Due to its nature as a protein, EGF has the drawback that it is very susceptible to factors that impair its stability, such as temperature and interaction with other agents, in particular proteases. There is thus need for EGF alternatives that exhibit, compared to EGF, improved (proteolytic and temperature) stability while retaining high affinity binding to and activation of EGFR.

Plants are a rich source of bioactive compounds with great values for drug design and development. However, plant peptides and proteins are often underexplored due to the general perception of instability.

The present invention meets this need by providing hyper-stable, plant-derived EGFR ligands that are useful for various applications, including skin and wound applications (including wound healing, skin and tissue regeneration) as well as alternative cell culture supplements for cell expansion and improving cell survival and cellular agricultural/food applications, including cultured meat.

### SUMMARY OF THE INVENTION

The present invention is based on the inventors' identification of novel cysteine-rich peptides (CRPs) derived from the plant Pereskia bleo that show EGFR-binding activity and high stability. Cysteine-rich peptides (CRPs) in general are small peptides characterized by high cysteine content, cross-linked by disulfide bridges. Their conformational-constrained structures and cysteine-rich core provide them with comparably high stability. The medicinal plant Pereskia bleo has been found to be a rich source of cysteine-rich peptides, which are collectively named as bleogens. The prototypic and cationic bleogen pB1 is a heparin-binding CRP with 36 residues, of which, five are Lys/Arg and six are Cys with a cysteine motif of C(X)₆C(X)₇CC(X)₃C(X)₁₀C (SEQ ID NO:2). Structurally different from the EGFR ligands in that it has a different and far more compact structure than the canonical EGF-like domain. Bleogen pB1 adopts a four loop structure with its disulfide linkages arranged in cystine-knot connectivity (Cys I-IV, Cys IIV, and Cys III-VI). Bleogen pB1 is biosynthesized as a two-domain precursor and the mature domain is released upon signal peptidase cleavage. Bleogen pB1 possesses a cation-polar-cation motif that contributes to its heparin-binding properties. Bleogen pB1 also shares sequence homology to the knottin-type anti-microbial peptides and exerts anti-Candida properties. Bleogen pB1 thus represents a first-in-class EGFR agonist, 59 years following the discovery of EGF.

In a first aspect, the present invention thus relates to an isolated peptide having EGFR binding activity, said peptide comprising or consisting of
(i) the amino acid sequence as set forth in SEQ ID NO:1;
(ii) an amino acid sequence that shares at least 60, preferably at least 70, more preferably at least 80, most preferably at least 90% sequence identity with the amino acid sequence set forth in SEQ ID NO:1 over its entire length;
(iii) an amino acid sequence that shares at least 80, preferably at least 90, more preferably at least 95% sequence homology with the amino acid sequence set forth in SEQ ID NO:1 over its entire length; or
(iv) a fragment of any one of (i)-(iii).

The peptide consisting of SEQ ID NO:1 is also referred to herein as “bleogen pB1” or “pB1”.

In another aspect, the present invention also relates to nucleic acid molecules encoding the peptides described herein, as well as a vector containing such a nucleic acid, in particular a copying vector or an expression vector.

In a further aspect, the invention is also directed to a host cell, preferably a non-human host cell, containing a nucleic acid as contemplated herein or a vector as contemplated herein. The host cell may be a bacterial cell, such as E. coli, or plant cell.

A still further aspect of the invention is a method for manufacturing a peptide as described herein, comprising culturing a host cell contemplated herein; and isolating the peptide from the culture medium or from the host cell. Another aspect is directed to a method for manufacturing a peptide as described herein by chemical synthesis, such as solid-phase peptide synthesis.

In a still further aspect, the present invention relates to compositions comprising the peptides described herein, in particular pharmaceutical, cosmetic or cosmeceutical compositions. Said compositions may additionally comprise a carrier and/or excipient.

In still another aspect, the invention relates to the use of peptides described herein or the compositions containing them for EGFR activation, in particular in ex vivo applications, such as cell culture.

In still another aspect, the invention is directed to one or more peptides of the invention or the compositions of the invention for use in a method for preventing or treating an EGF- or EGFR-related disease or disorder in a subject in need thereof. This aspect also covers uses of the peptides or cosmetic/pharmaceutical/cosmeceutical compositions of the invention for the manufacture of a medicament for the treatment or prevention of EGF- or EGFR-related diseases or disorders in a subject in need thereof, wherein said prevention or treatment may comprise administering a cosmetically, therapeutically or prophylactically effective amount of the peptides or compositions of the invention.

In a further aspect, the invention is directed to a method for the treatment or prevention of an EGF- or EGFR-related disease or disorder in a subject in need thereof comprising administering a cosmetically, prophylactically or therapeutically effective amount of one or more compounds of the invention or the composition of the invention to said subject.

### BRIEF DESCRIPTION OF THE DRAWINGS

FIG. 1. Sequence and structural comparison of bleogen pB1 and the seven related mammalian EGFR agonists. (A) The leaves of Pereskia bleo; (B) Precursor architecture, primary sequence, and disulfide connectivity of bleogen pB1; (C) WebLogo displaying the sequence comparison of all seven related mammalian EGFR agonists (EGF, TGFα, hb-EGF, betacellulin, amphiregulin, epiregulin, and epigen); the YXGXR motif (X, any amino acid) is shaded in red. (D) Overlay of loop 4 of bleogen pB1 with loop C of EGF (PDB entry: 1P9J), epiregulin (PDB entry: 1 K36), and TGFα (PDB entry: 1YUF); (E) Modeling interaction between bleogen pB1 and the extracellular domain of EGFR (PDB entry: 1IVO) using ClusPro Version 2.0 server.

FIG. 2. Chemical synthesis of bleogen pB1. (A) Synthesis scheme of bleogen pB1 and biotinpB1 by stepwise solid-phase method using Fmoc chemistry on Wang resin. Bleogen pB1 was synthesized using stepwise solid-phase Fmoc chemistry on Wang resin to yield PG-pB1 (PG: Protecting Group). Biotin-pB1 was synthesized by coupling Fmoc-Lys(biotin) to the N-terminus of PG-pB1 on resin. After TFA cleavage, the assembled linear precursor released from the resin support was immediately subjected to oxidative folding in 0.1 M ammonium bicarbonate at pH 8.0 and 10% dimethyl sulfoxide (DMSO) with a mixture of redox reagents cysteamine/cystamine of 10:1 molar ratio for 1 h; (B) Elution of natural and synthetic bleogen pB1 using RP-HPLC; (C) HPLC chromatogram of natural and synthetic bleogen pB1 using heparin-affinity chromatography.

FIG. 3. Bleogen pB1 exerts EGF-like activities. (A, B) Natural and synthetic bleogen pB1 promotes HaCaT cell proliferation for 72 h in a dose-dependent manner using crystal violet assay, with EGF as the positive control; All results were expressed as mean±S.D. from three independent experiments; (C) Bleogen pB1 promotes primary human keratinocyte proliferation for 72 h using crystal violet assay, with EGF as the positive control; All results were expressed as mean±S.D. from three independent experiments; *p<0.05 compared to control; (D) Bleogen pB1 promotes DNA synthesis in Ha-CaT cells in vitro using an EdU incorporation assay, with EGF as the positive control; All results were expressed as mean±S.D. from three independent experiments; *p<0.05 compared to control group. (E) Subcutaneous injection of bleogen pB1 (3 mg/kg, 10 mice) for five consecutive days accelerates incisor eruption in newborn mice by visual inspection from 153 h (saline control, 10 mice) to 125 h, whereas positive control EGF (3 mg/kg, 10 mice) accelerates incisor eruption to 100 h; Incisor eruption was defined as the time at which a given tooth first pierced the oral epithelium; n=10 mice per group; All results were expressed as mean±S.D.; *p<0.05 compared to control group. (F) Topical application of bleogen pB1 (10 nmol/wound; n=10 wounds; 5 mice) and EGF (1 nmol/wound; n=10 wounds; 5 mice) for three consecutive days post-injury accelerates wound healing compared to saline vehicle control group (n=30 wounds; 15 mice) in 5-mm full thickness splinted excisional wound model using C57 mice (Total 50 wounds; 25 mice). Top panel: schematic illustration of the treatment regimen. Bottom panel: percentage of wound course during the 14 day course post-injury. All results were expressed as mean±S.D.; *p<0.05 for all-treated groups compared to saline control group.

FIG. 4. Bleogen pB1 binds to EGFR. (A) Representative western blot image of biotin-pB1, biotinaB1 (negative control), and biotin-EGF (positive control) pull-down experiments of EGFR from HaCaT cell lysate using anti-EGFR mAb; N=three independent experiments; (B) Anti-EGFR neutralizing mAb (clone LA1) blocked the proliferative effects of bleogen pB1 in HaCaT cells using crystal violet assays. Mouse IgG1 was used as a control; All results were expressed as mean±S.D. from three independent experiments; *p<0.05 compared to pB1-treated group.

FIG. 5. Bleogen pB1 activates EGFR and its downstream signaling pathways. (A) Representative western blot analysis of immune-precipitated samples using EGFR antibody (magnetic beads conjugated) on phosphorylated-tyrosine (P-TYR) and EGFR in HaCaT cells following the incubation with 5 μM bleogen pB1; N=three independent experiments; (B) Representative western blot analysis on the phosphorylated MEK1/2 (p-MEK1/2), total MEK (T-MEK1/2), phosphorylated ERK1/2 (p-ERK1/2), and total ERK1/2 (T-ERK1/2) expressions in HaCaT cells following incubation with 5 μM bleogen pB1; N=three independent experiments; (C, D) Effects of AG1478 (a EGFR-specific tyrosine kinase inhibitor) and U0126 (a MEK-specific inhibitor) on bleogen pB1-induced HaCaT cell proliferation in serum-free medium for 72 h. All results were expressed as mean±S.D. from three independent experiments; *p<0.05 compared to pB1-treated group. (E) Incubation of bleogen pB1 for 6 h increased the luciferase activity in stably transfected SRE-luciferase reporter HaCaT cells. EGF was used as positive control. All results were expressed as mean±S.D. from three independent experiments; *,# p<0.05 compared to control group. (F) Bleogen pB1 treatment for 2 h upregulated the gene expressions of c-fos and c-Jun in HaCaT cells. All results were expressed as mean±S.D. from three independent experiments; *, # p<0.05 compared to control group.

FIG. 6. Positional scanning of the YAGQK region in bleogen pB1 using D-amino acid. (A) TR-FRET-based competitive displacement of biotin-EGF from EGFR using different concentrations of bleogen pB1, D-analogs ([Y25y]pB1, [A26a]pB1, [Q28q]pB1, [K29k]pB1), aB1 (negative control), rT7 (negative control) or EGF (positive control). All results were expressed as mean±S.D. from three independent experiments; (B) Proliferative effects of 1 μM bleogen pB1 or D-analogs ([Y25y]pB1, [A26a]pB1, [Q28q]pB1, [K29k]pB1) using HaCaT cell for 72 h using crystal violet assay. All results were expressed as mean±S.D. from three independent experiments; *p<0.05 compared to control group. # p<0.05 compared to pB1 group. (C) Anti-EGFR neutralizing mAb (clone LA1) blocked the proliferative effects of [K29k]pB1 in HaCaT cells using crystal violet assay. Mouse IgG1 was used as a control; All results were expressed as mean±S.D. from three independent experiments; # p<0.05 compared to [K29k]pB1 group. * p<0.05 compared to [K29k]pB1 with anti-EGFR mAb group.

FIG. 7. Bleogen pB1 and [K29k]pB1 accelerates wound healing in streptozotocin-induced diabetic mice. Topical application of bleogen pB1 (1 nmol/wound; n=12 wounds; 6 mice), [K29k]pB1 (1 nmol/wound; n=12 wounds; 6 mice), and EGF (1 nmol/wound; n=12 wounds; 6 mice) for five consecutive days post-injury, accelerates wound healing compared to saline vehicle control group (n=12 wounds; 6 mice) in 5-mm full thickness splinted excisional wound model using STZ-induced diabetic C57 mice (Total 48 wounds; 24 mice). Top panel: schematic illustration of the treatment regimen. Bottom panel: percentage of wound closure during the 14-day course, post-injury. All results were expressed as mean±S.D.; *p<0.05 for all-treated groups compared to saline control group.

FIG. 8. Bleogen pB1 and [K29k]pB1 are hyperstable EGFR agonist. Stability of bleogen pB1, [K29k]pB1, EGF, and S-alkylated pB1 (iodoacetamido-) under (A) heat (100° C.), (B) human serum, (C) pepsin, (D) trypsin, (E) pronase, and (F) neutrophil elastase treatment as analyzed by RP-HPLC; All results were expressed as mean±S.D. from three independent experiments; N.D.: non-detected.

FIG. 9. Mass spectrometry profiles for the removal of MBP from MBP-pB1 fusion protein using enterokinase enzyme.

FIG. 10. Co-elution of natural and recombinant bleogen pB1 by reverse phase HPLC (RP-HPLC).

### DETAILED DESCRIPTION

The present invention is based on the inventors' identification of novel cysteine-rich peptides (CRPs) having EGFR-binding activity isolated from Pereskia bleo. Specifically, the inventors successfully identified a novel hyper-stable EGFR ligand from Pereskia bleo named Bleogen pB1. The prototypic and cationic bleogen pB1 is a heparin-binding CRP with 36 residues, of which, five are Lys/Arg and six are Cys with a cysteine motif of C(X)₆C(X)₇CC(X)₃C(X)₁₀C (SEQ ID NO:2). Structurally different from the previously known EGFR ligands, bleogen pB1 adopts a four loop structure with its disulfide linkages arranged in cystine-knot connectivity (Cys I-IV, Cys II-V, and Cys III-VI). Bleogen pB1 is biosynthesized as a two-domain precursor and the mature domain is released upon signal peptidase cleavage. Bleogen pB1 possesses a cation-polar-cation motif that contributes to its heparin-binding properties. Bleogen pB1 also shares sequence homology to the knottin-type anti-microbial peptides and exerts anti-Candida properties.

The inventors further found that Bleogen pB1 does not contain an EGF-like domain and thus represents a first-in-class EGFR agonist, 59 years following the discovery of EGF. The 36-residue bleogen pB1 is the smallest naturally-occurring peptidyl EGFR agonist reported to date. It is 10- and 16-residues shorter than the two smallest EGFR agonists, the 46-residue epiregulin and the 52-residue TGF-α, respectively. EGF and TGF-α are classified as high-affinity EGFR agonists whereas amphiregulin and epiregulin as low-affinity agonists. Receptor displacement assays showed that bleogen pB1 is approximately 50-100-fold less potent than EGF, placing it as a low-affinity EGFR agonist. Similar to EGF, bleogen pB1 promotes keratinocyte proliferation, keratinocyte migration. The loop 4 of bleogen pB1 shares high sequence identity and structure similarity to the loop C of TGF-α. The presence of a conserved YXGXK/R (SEQ ID NO:3) motif indicates that they have a common “hot spot” for EGFR interaction.

Previous studies (U.S. Pat. No. 5,182,261; Tam J & Ke X (1989) Systematic approach to study the structure-activity of transforming growth factor α. Peptides: chemistry and biology [Proceedings of the 11th American Peptide Symposium], (ESCOM, Leiden), pp 75-77) have shown that mutations, particularly Y38 and R42 in the YXGXR loop C of TGF-α, resulted in a substantial decrease in both its EGFR affinity and EGF-like mitogenic potency in A431 cells. Likewise, in a separate study on EGF, its Y37 and R41 mutated analogs showed decrease in their EGF-like activities (Ogiso et al. (2002) Crystal structure of the complex of human epidermal growth factor and receptor extracellular domains. Cell 110(6):775-78746, 62-65). Collectively, these findings suggest the importance of the YXGXR motif as the putative receptor-contacting site for EGFR agonists.

By point-substituting the YAGQK motif in Bleogen pB1, it could be shown that there is a decrease in the EGF-like biological activities for all Ala-substituted pB1 analogs, consistent with the observations for EGF and TGF-α. In contrast, two D-amino acid-substituted pB1 analogs, K29k pB1 and Y25y pB1 showed increased EGF-like biological activities. By replacing Lys at position 29 with the D-Lys in K29k pB1, the low-affinity bleogen pB1 was transformed to a high-affinity EGFR agonist. K29k pB1 was found to be as potent as EGF with a 60-fold improved potency compared to bleogen pB1. The in vitro results were supported by the in vivo wound healing STZ-diabetic mouse model which showed that the effects of K29k pB1 and EGF were comparable.

Sequence alignment showed that K29 of bleogen pB1 corresponds to R41 of EGF and R42 of TGF-α. Arg at this position is a key residue that is absolutely conserved across all known canonical EGFR agonists (Ogiso, supra). Structural and mutational studies have also identified the R41 residue of EGF to be critical for the formation of a salt bridge with D355 of EGFR, essential for receptor binding (Ogiso, supra). In agreement with previous mutational studies on TGF-α and EGF, these results showed that K29 is an important molecular determinant for the EGF-like activities of bleogen pB1.

Comparing to an EGFR agonist, bleogen pB1 shares similar functional characteristics, but differs in its primary sequence, secondary and tertiary structure, and biosynthesis. All known agonists of the EGF family, particularly those from mammalian origin, contain an EGF-like domain (Singh, Carpenter & Coffey (2016) EGF receptor ligands: recent advances. F1000Research 5). In addition, mammalian EGFR agonists are biosynthesized as type 1 transmembrane precursors and are released upon proteolytic cleavage by a disintegrin and metalloproteinases (ADAMs). In contrast, bleogen pB1 does not contain an EGF-like domain; instead, it contains a cysteine motif of SEQ ID NO:2, typical of the 6-cysteine-hevein-like peptide family, and is biosynthesized as a two-domain precursor consisting of an ER signal peptide and a mature domain. This cysteine motif is a cystine-knot disulfide connectivity that is more resistant to proteolytic degradation than EGF. Thus, bleogen pB1 is a prototypic member of a new class of hyperstable EGFR agonists with a non-cannonical primary sequence, secondary and tertiary structure, biosynthetic pathway, and high proteolytic stability.

Significant efforts have been made to extend the half-life of EGF through the development of polymer-based system, encapsulation, and nanotechnology. It was found that Bleogen pB1 and the potent K29k pB1 displays EGF-like activities and share a structurally compact scaffold which is at least 100-fold more stable than EGF against proteolytic degradation. Substituting the Lys from its L- to D-form yielded K29k pB1 with comparable effects to EGF. By further developing current methodologies, it is feasible to scale-up the production of modified peptides like K29k pB1. Utilizing orthogonal tRNA/synthetase pairing eases the incorporation of unnatural amino acids for biosynthetic production in E. coli. (Liu & Schultz (2010) Adding new chemistries to the genetic code. Annu. Rev. Biochem. 79:413-444; Liu et al. (2012) Genetic incorporation of d-lysine into diketoreductase in Escherichia coli cells. Amino Acids 43(6):2553-2559). Besides, the highly time and cost efficient chemical synthesis and fragment condensation approaches can be employed for industrial manufacturing. Marketed examples using these techniques are bivalirubin, a thrombin inhibitor, and T-20, a HIV fusion inhibitor. In conclusion, the discovery of bleogen pB1, a hyperstable, non-canonical, and the smallest plant-derived EGFR agonist provides a promising lead. The discovery of an improved pB1 analog K29k pB1, which is equally potent as EGF, could further advance the development of potent therapeutic analogs for wound healing, regenerative medicine, and skincare.

Based on the above findings, the invention, in a first aspect, covers peptides having activity in isolated form and, more specifically, is directed to an isolated peptide comprising, consisting essentially of or consisting of the amino acid sequence as set forth in SEQ ID NO:1 (Bleogen pB1).

The peptide consisting of the amino acid sequence set forth in SEQ ID NO:1 is also referred to as “Bleogen pB1” or “pB1” herein. “Isolated”, as used herein, relates to the peptide in a form where it has been at least partially separated from other cellular components it may naturally occur or associate with. The peptide may be a recombinant peptide, i.e. peptide produced in a genetically engineered organism that does not naturally produce said peptide.

A peptide according to the present invention exhibits EGFR binding activity, i.e. it is capable of recognizing and binding EGFR in a specific manner, i.e. binds it preferentially over other receptor types, typically with at least 10-fold or 100-fold higher affinity than those observed for non-specific binding. Furthermore, the peptides preferably also exhibit EGFR activing activity, i.e. function as EGFR agonists. In various preferred embodiments, the EGFR binding is similar to that of EGF, i.e. the affinity is within ±50% of that of EGF.

“Peptide”, as used herein, relates to polymers made from amino acids connected by peptide bonds. The peptides, as defined herein, can comprise 10 or more amino acids, preferably 20 or more, more preferably 25 or more amino acids, for example 25 to 50 amino acids, more preferably 30 to 40 or 32 to 36 amino acids. “Polypeptide”, as used herein, relates to peptides comprising more than 100 amino acids.

In various embodiments, the peptide comprises or consists of an amino acid sequence that is at least 60%, 65%, 70%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.25%, or 99.5% identical or homologous to the amino acid sequence set forth in SEQ ID NO:1 over its entire length. Alternatively, the identity or homology may be relative to any one of SEQ ID Nos. 12-14. In some embodiments, it has an amino acid sequence that shares at least 60, preferably at least 70, more preferably at least 80, most preferably at least 90% sequence identity with the amino acid sequence set forth in SEQ ID NO:1, 12, 13 or 14 over its entire length or has an amino acid sequence that shares at least 80, preferably at least 90, more preferably at least 95% sequence homology with the amino acid sequence set forth in SEQ ID NO:1, 12, 13 or 14 over its entire length.

In various embodiments, the peptide may be a precursor of the mature enzyme. In such embodiments, it may comprise additional amino acid sequences besides those set forth in SEQ ID NO:1, 12, 13 or 14. Such precursors may typically comprise an N-terminal signal peptide, typically of 20-30 amino acids in length, that may be cleaved during posttranslational processing.

The identity of nucleic acid sequences or amino acid sequences is generally determined by means of a sequence comparison. This sequence comparison is based on the BLAST algorithm that is established in the existing art and commonly used (cf. for example Altschul et al. (1990) “Basic local alignment search tool”, J. Mol. Biol. 215:403-410, and Altschul et al. (1997): “Gapped BLAST and PSI-BLAST: a new generation of protein database search programs”; Nucleic Acids Res., 25, p. 3389-3402) and is effected in principle by mutually associating similar successions of nucleotides or amino acids in the nucleic acid sequences and amino acid sequences, respectively. A tabular association of the relevant positions is referred to as an “alignment.” Sequence comparisons (alignments), in particular multiple sequence comparisons, are commonly prepared using computer programs which are available and known to those skilled in the art.

A comparison of this kind also allows a statement as to the similarity to one another of the sequences that are being compared. This is usually indicated as a percentage identity, i.e. the proportion of identical nucleotides or amino acid residues at the same positions or at positions corresponding to one another in an alignment. The more broadly construed term “homology”, in the context of amino acid sequences, also incorporates consideration of the conserved amino acid exchanges, i.e. amino acids having a similar chemical activity, since these usually perform similar chemical activities within the protein. Conservative amino acid substitutions include without limitation: Conservative amino acid substitutions in the context of the invention encompass, for example, G=A=S, I=V=L=M, D=E, N=Q, K=R, Y=F, S=T, A=I=V=L=M, Y=F=W. The similarity of the compared sequences can therefore also be indicated as a “percentage homology” or “percentage similarity.” Indications of identity and/or homology can be encountered over entire (poly)peptides or genes, or only over individual regions. Homologous and identical regions of various nucleic acid sequences or amino acid sequences are therefore defined by way of matches in the sequences. Such regions often exhibit identical functions. They can be small, and can encompass only a few nucleotides or amino acids. Small regions of this kind often perform functions that are essential to the overall activity of the protein. It may therefore be useful to refer sequence matches only to individual, and optionally small, regions. Unless otherwise indicated, however, indications of identity and homology herein refer to the full length of the respectively indicated nucleic acid sequence or amino acid sequence.

In various embodiments, the peptide described herein comprises the amino acid residues C at any one or more of the positions corresponding to positions 2, 9, 17, 18, 22 and 33 of SEQ ID NO:1, preferably at least in positions 2 and 18, 9 and 22 and/or 17 and 33, more preferably two sets of the previous pairs, most preferably in all six positions.

In various embodiments, at least the cysteine-rich core motif C(X)ₙC(X)ₘCC(X)ₒC(X)ₚC, wherein X can be any amino acid with the exception of C, n is an integer from 4 to 8, m is an integer from 5 to 9, o is an integer from 1 to 5, and p is an integer from 8 to 12, preferably C(X)₆C(X)₇CC(X)₃C(X)₁₀C (SEQ ID NO:2); and/or the amino acid sequence motif YXGXK/R (SEQ ID NO:3), wherein X can be any amino acid with the exception of C, are present, preferably in combination.

In various embodiments, the amino acid motif YXGXK/R (SEQ ID NO:3) may be included in the (X)ₚ part of the motif C(X)ₙC(X)ₘCC(X)ₒC(X)ₚC or the (X)₁₀ part of the motif of SEQ ID NO:2. Accordingly, in various embodiments, the peptide of the invention comprises the amino acid sequence

(SEQ ID NO: 4)
C(X)₆C(X)₇CC(X)₃CXXYXGXK/RXXXC,
(SEQ ID NO: 5)
CK/RPXGXK/RCXXXXXPPCCXXXCXK/RYXGXK/RXGXCXXK/R,
or
(SEQ ID NO: 6)
CKPXGXKCXEXXXPPCCXXXCXRYXGXKXGXCXXR.

The motif of SEQ ID NO:3 may be YAGXK (SEQ ID NO:15), wherein X can be any amino acid with the exception of C, preferably D, E, N or Q, more preferably N or Q, most preferably Q. The motif of SEQ ID NO:3 may also be YXGQK (SEQ ID NO:16), wherein X can be any amino acid with the exception of C, preferably A, V, L, S, more preferably A, V or S, most preferably A. These embodiments also encompass peptides wherein the motif of SEQ ID NO:15 or 16 is included in the C-rich motif or SEQ ID NO:2, 4, 5 or 6, as defined above.

In various embodiments, the amino acids given in SEQ ID Nos. 2-6 and 15-16 are retained in the backbone of SEQ ID NO:1, while the remaining amino acids may be varied within the given ranges of sequence identity and/or homology. In these embodiments said substitutions may include substitutions of any other amino acid to C.

In various embodiments, the peptide comprises the amino acids corresponding to residues 2-33 of SEQ ID NO:1. In various embodiments, the fragments of the invention comprise an amino acid sequence that corresponds to amino acids 2-33 of SEQ ID NO:1. The fragments are thus preferably at least 32 amino acids in length. Other fragments encompassed by the present invention are those that correspond to N- and/or C-terminally truncated versions of SEQ ID NO:1 or corresponding sequences. N-terminal truncated fragments preferably lack only the first N-terminal amino acid using the positional numbering of SEQ ID NO:1, while C-terminally truncated fragments may lack 1-3 amino acids from the C-terminal end using the positional numbering of SEQ ID NO:1. It may in various embodiments be advantageous that the amino acid corresponding to position 36 of SEQ ID NO:1 is present.

In various embodiments, the peptides of the invention differ from the sequence set forth in SEQ ID NO:1 in that they comprise one more amino acid variations selected from insertions, deletions and/or substitutions. In various embodiments, these variants of SEQ ID NO:1 comprise any one or more of SEQ ID Nos. 2-6 and 15-16. In various embodiments, the peptides of the invention comprise one or more amino acid substitutions. These substitutions may comprise substitution of one naturally occurring L-amino acid by another L-amino acid, preferably selected as disclosed herein, in particular for the positions designated as X in any one of SEQ ID Nos. 2-6 and 15-16. In other embodiments, the substitution is a substitution of an L-amino acid by a D-amino acid, typically the corresponding D-amino acid. The peptide may thus comprise one or more D-amino acids. In various embodiments, these substitutions comprise Y25y and/or K29k, most preferred is a variant comprising the K29k substitution. The amino acid sequences of such variants are set forth in SEQ ID Nos. 7-9. As defined below, capital letters mean L-amino acids in the one letter code, while small letters mean D-amino acids in the one letter code. “K” is thus L-lysine while “k” is D-lysine. “K29k” thus means the replacement of L-lysine (K) at a position corresponding to position 29 in SEQ ID NO:1 by D-lysine (k). All positional numbering as used herein refers, if not indicated otherwise, to the positional numbering using SEQ ID NO:1 for reference.

In various embodiments, the peptide may have a positive net charge. This means that the peptide comprises amino acid residues such that the sum of all positive charges of positively charged amino acids (H, K, R) is higher than the sum of all negative charges of negatively charged amino acids (D, E). In other words, the sum of the amino residues of H, K and R present in the peptide is higher than the sum of amino acid residues D and E.

In various embodiments, the peptide comprises one, two or three, preferably three, disulfide bridges, preferably selected from disulfide bridges between C2 and C18, C9 and C22, and C17 and C33, using the positional numbering of SEQ ID NO:1.

Compared to wild type EGF, the amino acid sequence of which is set forth in SEQ ID NO:10, the peptides of the invention have, in various embodiments, an at least 2-fold, preferably at least 5-fold, more preferably at least 10-fold higher stability toward heat and/or proteases. These may be determined using conventional assays known to those skilled in the art, where EGF and the peptide of the invention are assayed under identical conditions.

All amino acid residues are generally referred to herein by reference to their one letter code and, in some instances, their three letter code. This nomenclature is well known to those skilled in the art and used herein as understood in the field. Substitutions are referred to by the starting amino acid, position number and target amino acid. For example, “K29A” means that K in position 29 is exchanged to A. In addition, all amino acids referred to herein with capital letters or without any further indication are L-amino acids. D-amino acids are referred to herein in small letters using the one letter code. “K29k” thus means that L-lysine in position 29 is replaced by D-lysine. Furthermore, in any sequence given herein amino acids separated by “/” means that those can be present in the alternative. “K/R” thus means that at the given position a K or R residue may be present.

In addition to the above-described modifications, peptides according to the embodiments described herein can comprise amino acid modifications, in particular amino acid substitutions, insertions, or deletions. Such peptides are, for example, further developed by targeted genetic modification, i.e. by way of mutagenesis methods, and optimized for specific purposes or with regard to special properties (for example, with regard to their activity, stability, etc.). If such additional modifications are introduced into the peptides of the invention, these preferably do not affect, alter or reverse the sequence motifs detailed above, i.e. the C-rich motifs (with the exception of the variable residues X) and the YXGXK/R loop. This means that the above-defined fixed positions of these residues/motifs are not changed by these additional mutations beyond that what is defined above.

In various embodiments, the peptides of the invention may be post-translationally modified, for example glycosylated. Such modification may be carried out by recombinant means, i.e. directly in the host cell upon production, or may be achieved chemically or enzymatically after synthesis of the peptide, for example in vitro.

The objective of the described modifications may be to introduce targeted mutations, such as substitutions, insertions, or deletions, into the known molecules in order, for example, to increase binding specificity/affinity and/or improve the activity. For this purpose, in particular, the surface charges and/or isoelectric point of the molecules, and thereby their interactions with the target, can be modified. Alternatively or additionally, the stability of the peptide can be enhanced by way of one or more corresponding mutations. Advantageous properties of individual mutations, e.g. individual substitutions, can supplement one another. Examples of such modifications have been described above and include replacement of selected amino acids by their D-amino acid counterparts, in particular the K29k variant.

In various embodiments, the peptide may be characterized in that it is obtainable from a peptide as described above as an initial molecule by single or multiple conservative amino acid substitution. The term “conservative amino acid substitution” means the exchange (substitution) of one amino acid residue for another amino acid residue, where such exchange does not lead to a change in the polarity or charge at the position of the exchanged amino acid, e.g. the exchange of a nonpolar amino acid residue for another nonpolar amino acid residue. Conservative amino acid substitutions in the context of the invention encompass, for example, the substitutions disclosed above.

Alternatively or additionally, the peptide may be characterized in that it is obtainable from a peptide contemplated herein as an initial molecule by fragmentation or by deletion, insertion, or substitution mutagenesis, and encompasses an amino acid sequence that matches the initial molecule as set forth in SEQ ID Nos. 1, 12-14 over a length of at least 32 continuously connected amino acids that correspond to amino acids 2-33 of SEQ ID NO:1. It is preferred that in such embodiments, the amino acids C2, C9, C17, C18, C22 and C33 as well as Y25, G27 and K29 are still present either in their native L-conformation or artificially introduced D-form.

In various embodiments, the present invention thus also relates to fragments of the peptides described herein, with said fragments retaining the desired binding and activity. It is preferred that they have at least 50%, more preferably at least 70, most preferably at least 90% of the affinity and/or activity of the initial molecule, preferably of the peptide having the amino acid sequence of SEQ ID NO:1. Preferred fragments have already been defined above.

The nucleic acid molecules encoding the peptides described herein, as well as a vector containing such a nucleic acid, in particular a copying vector or an expression vector also form part of the present invention.

These can be DNA molecules or RNA molecules. They can exist as an individual strand, as an individual strand complementary to said individual strand, or as a double strand. With DNA molecules in particular, the sequences of both complementary strands in all three possible reading frames are to be considered in each case. Also to be considered is the fact that different codons, i.e. base triplets, can code for the same amino acids, so that a specific amino acid sequence can be coded by multiple different nucleic acids. As a result of this degeneracy of the genetic code, all nucleic acid sequences that can encode one of the above-described peptides are included in this subject of the invention. The skilled artisan is capable of unequivocally determining these nucleic acid sequences, since despite the degeneracy of the genetic code, defined amino acids are to be associated with individual codons. The skilled artisan can therefore, proceeding from an amino acid sequence, readily ascertain nucleic acids coding for that amino acid sequence. In addition, in the context of nucleic acids according to the present invention one or more codons can be replaced by synonymous codons. This aspect refers in particular to heterologous expression of the peptides contemplated herein. For example, every organism, e.g. a host cell of a production strain, possesses a specific codon usage. “Codon usage” is understood as the translation of the genetic code into amino acids by the respective organism. Bottlenecks in protein biosynthesis can occur if the codons located on the nucleic acid are confronted, in the organism, with a comparatively small number of loaded tRNA molecules. Also it codes for the same amino acid, the result is that a codon becomes translated in the organism less efficiently than a synonymous codon that codes for the same amino acid. Because of the presence of a larger number of tRNA molecules for the synonymous codon, the latter can be translated more efficiently in the organism.

By way of methods commonly known today such as, for example, chemical synthesis or the polymerase chain reaction (PCR) in combination with standard methods of molecular biology or protein chemistry, a skilled artisan has the ability to manufacture, on the basis of known DNA sequences and/or amino acid sequences, the corresponding nucleic acids all the way to complete genes. Such methods are known, for example, from Sambrook, J., Fritsch, E. F., and Maniatis, T, 2001, Molecular cloning: a laboratory manual, 3rd edition, Cold Spring Laboratory Press.

“Vectors” are understood for purposes herein as elements—made up of nucleic acids—that contain a nucleic acid contemplated herein as a characterizing nucleic acid region. They enable said nucleic acid to be established as a stable genetic element in a species or a cell line over multiple generations or cell divisions. In particular when used in bacteria, vectors are special plasmids, i.e. circular genetic elements. In the context herein, a nucleic acid as contemplated herein is cloned into a vector. Included among the vectors are, for example, those whose origins are bacterial plasmids, viruses, or bacteriophages, or predominantly synthetic vectors or plasmids having elements of widely differing derivations. Using the further genetic elements present in each case, vectors are capable of establishing themselves as stable units in the relevant host cells over multiple generations. They can be present extra-chromosomally as separate units, or can be integrated into a chromosome respectively into chromosomal DNA.

Expression vectors encompass nucleic acid sequences which are capable of replicating in the host cells, by preference microorganisms, particularly preferably bacteria, that contain them, and expressing therein a contained nucleic acid. In various embodiments, the vectors described herein thus also contain regulatory elements that control expression of the nucleic acids encoding a peptide of the invention. Expression is influenced in particular by the promoter or promoters that regulate transcription. Expression can occur in principle by means of the natural promoter originally located in front of the nucleic acid to be expressed, but also by means of a host-cell promoter furnished on the expression vector or also by means of a modified, or entirely different, promoter of another organism or of another host cell. In the present case at least one promoter for expression of a nucleic acid as contemplated herein is made available and used for expression thereof. Expression vectors can furthermore be regulated, for example by way of a change in culture conditions or when the host cells containing them reach a specific cell density, or by the addition of specific substances, in particular activators of gene expression. One example of such a substance is the galactose derivative isopropyl-beta-D-thiogalactopyranoside (IPTG), which is used as an activator of the bacterial lactose operon (lac operon). In contrast to expression vectors, the contained nucleic acid is not expressed in cloning vectors.

In a further aspect, the invention is also directed to a host cell, preferably a non-human host cell, containing a nucleic acid as contemplated herein or a vector as contemplated herein. A nucleic acid as contemplated herein or a vector containing said nucleic acid is preferably transformed into a microorganism, which then represents a host cell according to an embodiment. Methods for the transformation of cells are established in the existing art and are sufficiently known to the skilled artisan. All cells are in principle suitable as host cells, i.e. prokaryotic or eukaryotic cells. Those host cells that can be manipulated in genetically advantageous fashion, e.g. as regards transformation using the nucleic acid or vector and stable establishment thereof, are preferred, for example single-celled fungi or bacteria. In addition, preferred host cells are notable for being readily manipulated in microbiological and biotechnological terms. This refers, for example, to easy culturability, high growth rates, low demands in terms of fermentation media, and good production and secretion rates for foreign proteins. The peptides can furthermore be modified, after their manufacture, by the cells producing them, for example by the addition of sugar molecules, formylation, amination, etc. Post-translation modifications of this kind can functionally influence the peptide.

Further embodiments are represented by those host cells whose activity can be regulated on the basis of genetic regulation elements that are made available, for example, on the vector, but can also be present a priori in those cells. They can be stimulated to expression, for example, by controlled addition of chemical compounds that serve as activators, by modifying the culture conditions, or when a specific cell density is reached. This makes possible economical production of the proteins contemplated herein. One example of such a compound is IPTG, as described earlier.

Preferred host cells are prokaryotic or bacterial cells, such as E. coli cells. Bacteria are notable for short generation times and few demands in terms of culturing conditions. As a result, economical culturing methods resp. manufacturing methods can be established. In addition, the skilled artisan has ample experience in the context of bacteria in fermentation technology. Gram-negative or Gram-positive bacteria may be suitable for a specific production instance, for a wide variety of reasons to be ascertained experimentally in the individual case, such as nutrient sources, product formation rate, time requirement, etc. In various embodiments, the host cells may be E. coli cells.

Host cells contemplated herein can be modified in terms of their requirements for culture conditions, can comprise other or additional selection markers, or can also express other or additional proteins/peptides. They can, in particular, be those host cells that transgenically express multiple peptides.

The host cell can, however, also be a eukaryotic cell, which is characterized in that it possesses a cell nucleus. A further embodiment is therefore represented by a host cell which is characterized in that it possesses a cell nucleus. In contrast to prokaryotic cells, eukaryotic cells are capable of post-translationally modifying the protein/peptide that is formed. Examples thereof are fungi such as Actinomycetes, or yeasts such as Saccharomyces or Kluyveromyces or insect cells, such as Sf9 cells. This may be particularly advantageous, for example, when the peptides, in connection with their synthesis, are intended to experience specific modifications made possible by such systems. Among the modifications that eukaryotic systems carry out in particular in conjunction with protein synthesis are, for example, the bonding of low-molecular-weight compounds such as membrane anchors or oligosaccharides. In various embodiments, the host cells are thus eukaryotic cells.

The host cells contemplated herein are cultured and fermented in a usual manner, for example in discontinuous or continuous systems. In the former case a suitable nutrient medium is inoculated with the host cells, and the product is harvested from the medium after a period of time to be ascertained experimentally. Continuous fermentations are notable for the achievement of a flow equilibrium in which, over a comparatively long period of time, cells die off in part but are also in part renewed, and the peptide formed can simultaneously be removed from the medium.

Host cells contemplated herein are preferably used to manufacture the peptides described herein.

A further aspect of the invention is therefore a method for manufacturing/producing a peptide as described herein, comprising culturing a host cell contemplated herein under conditions that allow expression of the peptide; and isolating the peptide from the culture medium or from the host cell. Culture conditions and mediums can be selected by those skilled in the art based on the host organism used by resorting to general knowledge and techniques known in the art. For example, expression of the peptide may be carried out by using a fusion protein where the peptide of the invention is fused to another peptide/protein that facilitates expression/isolation/purification, for example by affinity chromatography. Such fusion constructs are typically processed by treatment with a site-specific protease that cleaves the expression/affinity tag and thus releases the peptide of interest.

In another aspect, the present invention relates to the use of the peptides disclosed herein as a pharmaceutical. The compounds of the invention are thus contemplated for use as a pharmaceutical.

In still another aspect, the invention is directed to one or more peptides of the invention for use in a method for preventing or treating an EGF- or EGFR-related disease, disorder or condition in a subject in need thereof. This aspect also covers uses of the peptides of the invention for the manufacture of a medicament for the treatment or prevention of an EGF- or EGFR-related disease, disorder or condition in a subject in need thereof, wherein said prevention or treatment may comprise administering a therapeutically or prophylactically effective amount of the peptides of the invention. “EGF- or EGFR-related disease, disorder or condition” include diseases/disorders and conditions that are directly caused by aberrant activities of EGF/EGFR in a subject, but also include diseases, disorders and conditions in which EGF and EGFR play a role insofar as the signaling pathway is involved and that would benefit from EGF/EGFR activation. Such conditions include, without limitation, wound healing, ulcers, and neurodegenerative diseases, including but not limited to diabetic ulcers, gastric ulcers, esophageal ulcers, duodenal ulcers, burn wounds, surgical wounds, pressure wounds, chemotherapy-induced wounds, corneal wounds, Alzheimer's disease, as well as conditions of the skin. Skin-related applications include, without limitation, wrinkle improvement, skin hydration, pigmentation prevention, improvement of skin elasticity, differentiation of skin stem cells and the like. The above treatments thus include promoting wound healing in a subject or treating any of the mentioned conditions, such as ulcers and neurodegenerative diseases.

In a further aspect, the invention is directed to a method for the treatment or prevention of an EGF- or EGFR-related disease, disorder or condition in a subject in need thereof comprising administering a prophylactically or therapeutically effective amount of one or more peptides of the invention to said subject.

In general, peptides of the invention will be administered in therapeutically/prophylactically effective amounts via any of the usual and acceptable modes known in the art, either singly or in combination with one or more other therapeutic agents. A therapeutically/prophylactically effective amount may vary widely depending on the severity of the disease, the age and relative health of the subject, the potency of the peptide used and other factors.

The peptides of the invention can be administered as pharmaceutical compositions by any conventional route, in particular topically, e.g., in the form of lotions, gels, eye drops, ointments or creams, but also parenterally, e.g., in the form of injectable solutions or suspensions. Such applications also include dosage forms, such as bandaids, where the composition may be provided on carrier material, such as a textile or fibrous material, and hydrogels.

The invention thus also relates to a pharmaceutical composition comprising one or more peptide(s) of the invention and a pharmaceutically acceptable excipient or carrier. The carrier may include diluents and/or solvents.

Pharmaceutical compositions comprising a peptide of the present invention in free form or in a pharmaceutically acceptable salt form in association with at least one pharmaceutically acceptable carrier or diluent can be manufactured in a conventional manner by mixing, granulating or coating methods. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Peptides of the invention can be administered in therapeutically effective amounts in combination with one or more therapeutic agents (pharmaceutical combinations). Non-limiting examples of compounds which can be used in combination with compounds of the invention are known protease inhibitors, EGF and EGF agonists.

Where the peptides of the invention are administered in conjunction with other therapies, dosages of the co-administered compounds will of course vary depending on the type of co-drug employed, on the specific drug employed, on the condition being treated and so forth.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a peptide of the invention and a co-agent, are both administered to a patient simultaneously in the form of a single entity or dosage. The term “non-fixed combination” means that the active ingredients, e.g. a peptide of the invention and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

The pharmaceutical compositions may be used in a method for preventing or treating an EGF- or EGFR-related disease, disorder or condition in a subject in need thereof.

In a further aspect, the invention is directed to a method for the treatment or prevention of an EGF- or EGFR-related disease, disorder or condition in a subject in need thereof comprising administering a prophylactically or therapeutically effective amount of the pharmaceutical composition of the invention to said subject.

All embodiments described above for pharmaceutical compositions similarly apply to cosmetic or cosmeceutical compositions that also form part of the present invention. It is understood that all accompanying agents present in these compositions besides the peptides of the invention are then cosmetically or cosmeceutically acceptable. As the cosmetic/cosmeceutical compositions may typically be topical administration forms, the disclosure above in relation to topical forms of pharmaceutical compositions similarly applies to cosmetic/cosmeceutical compositions. “Cosmeceutic” and “cosmeceutically”, as used herein, relate to compositions that have mixed therapeutic and cosmetic effects or where those effects are not clearly distinguishable. Such cosmetic applications include applications in which the skin is involved, such as wrinkle improvement, skin hydration, pigmentation prevention, improvement of skin elasticity and the like.

The peptides of the invention may further be used for activating EGFR in a cell. Such uses may include ex vivo uses, such as in cell and tissue culture, including applications for agricultural or food purposes, such as cultured meat, as well as tissue engineering applications, such as tissue engineering of hard and soft tissues and osteochondral constructs.

All embodiments disclosed herein in relation to the peptides and nucleic acids are similarly applicable to the uses and methods described herein and vice versa.

The invention is further illustrated by the following non-limiting examples and the appended claims.

### EXAMPLES

### Materials and Methods

### Materials

All the chemicals and solvents, unless otherwise stated, were purchased from Sigma Aldrich, US, and Fisher Scientific, US. Bleogen pB1, as referred to in the following, has the amino acid sequence set forth in SEQ ID NO:1.

### Extraction and Purification of Natural Bleogen pB1

Fresh Pereskia bleo leaves were collected from the Nanyang Community Herb Garden at Nanyang Technological University, Singapore (courtesy of Mr. Ng Kim Chuan). Fresh leaves (1 kg) of Pereskia bleo were blended with water for 15 min and centrifuged at 9,000 rpm for 10 min at 4° C. (Beckman Coulter, US) and the supernatant was filtered through 1 μm pore size glass fiber filter paper (Sartorius, Singapore). The filtrate was then loaded onto a C18 flash column (Grace Davison, US) and eluted with 60% ethanol. The eluted fractions were then loaded onto an SP Sepharose resin column (GE Healthcare, UK), eluted with 1 M NaCl (pH 3.0), and followed by ultrafiltration (ViVaflow 200, 2000 MWCO hydrostat). Further purification was performed by RP-HPLC and heparin-affinity chromatography (Shimadzu, Japan). Matrix assisted laser desorption/ionization-Time of flight mass spectrometry (MALDI-TOF MS) was used to identify the presence of bleogen pB1 in the eluted fractions. The eluted fractions were lyophilized for storage at room temperature.

### In Silico Modeling

To model the interactions between the NMR structure of bleogen pB1 and the crystal structure of human EGFR (PDB entry: 1VIO), protein-protein docking server ClusPro Version 2.0 was used. The docking involves global rigid docking using fast Fourier transform correlation approach. Two sets of 900 lowest energy structures (using electrostatic energy, van der Waals attractions, and van der Waals repulsions) were retained. The second step included clustering the retained structure using pairwise RMSD. Minimizing energy of the complexes, the 10 largest clusters were refined. Clusters ranked the highest displayed the most contacts with the protein.

Solid-phase peptide synthesis and oxidative folding of bleogen pB1 Synthetic bleogen pB1 (SEQ ID NO:1), its Ala-analogs ([Y25A]pB1, [G27A]pB1, [Q28A]pB1, [K29A]pB1), and D-analogs ([Y25y]pB1 (SEQ ID NO:7), [A26a]pB1, [Q28q]pB1, [K29k]pB1 (SEQ ID NO:8)) were manually synthesized by Fmoc-based solid phase peptide synthesis on Wang resin at room temperature. The synthesized peptides were cleaved in a cleavage cocktail (92.5% TFA); 2.5% H₂O; 2.5% 1,2-ethanedithiol; 2.5% triisopropylsilan) at room temperature for 1 h. The crude cleaved products were then folded under the following folding conditions: 10% DMSO, 90% 0.1 M NH₄HCO₃ aq. with pH at 8.0, and cystamine (10 equivalent) and cysteamine (100 equivalent) for 1 h at room temperature. The folded pB1 and its Ala- and D-analogs were purified by preparative HPLC (250×21 mm, 5 μm) (Phenomenex, US) and identified using MALDI-TOF MS. A linear gradient of mobile phase A (0.1% TFA/H₂O) and mobile phase B (0.1% TFA/acetonitrile (ACN)) were used. The folding yield was calculated to be 70% by HPLC analyses. ¹H NMR, RP-H PLC, and heparin affinity chromatography were performed to demonstrate the integrity of synthetic bleogen pB1 as compared to its native form.

N-terminal biotin labeling of pB1, [Y25y]pB1, and [K29k]pB1 were performed on peptide resin with a mixture of Fmoc-lysin(biotin)-OH (4.0 eq.), N,N-diisopropylethylamine (DIPEA; 6.0 eq.), (benzotriazol-1-yloxy)tripyrrolidinophosphonium hexafluorophosphate (PyBOP; 4.0 eq.) in 50% dimethylformamide (DMF), and 50% 1-methyl-2-pyrrolidinone (NMP) for 2 h at room temperature. After 2 h of reaction, the biotin-labeled-pB1 was cleaved and oxidatively-folded in the conditions described previously. The folded biotin-pB1 was purified by preparative HPLC and identified using MALDI-TOF MS.

### Cell Culture and Transfection

HaCaT (human keratinocyte), HUVEC-CS (endothelial cells) cells were cultured in Dulbecco's modification of Eagle's medium (DMEM) supplemented with 10% fetal bovine serum and 100 U/mL of penicillin and streptomycin. Normal Human Epidermal Keratinocytes adult purchased from Lifeline® cell technology were cultured in Lifeline® DermaLife K Medium. They were grown in a 5% CO₂ humidified incubator at 37° C. HaCaT cells were stably transfected with pGL4.33[luc2P/SRE/Hygro] (Promega) and selected using 250 μg/mL hygromycin. Stable HaCaT cells expressing SRE luciferase reporter were maintained with 250 μg/mL hygromycin.

### Cell Proliferation Assay

Cell proliferation was determined using crystal violet staining. Briefly, 1.0×10⁴ HaCaT cells per well were seeded in a 96-well plate with bleogen pB1 or EGF (positive control) or 3417-Da-peptide aB1 (negative control) in serum-free medium. 3417-Da-peptide aB1 is a CRP isolated from Achyranthes bidentata with the amino acid sequence NCESGTSCIPGAQHNCCSGVCVPIVTIFYGVCY (SEQ ID NO:11). Crystal violet staining was performed as previously described. Briefly, after the incubation period, the wells were fixed with 4% buffered paraformaldehyde for 20 min. The cells were then stained with 0.25% crystal violet in 20% methanol for 15 min. The excess crystal violet stain was rinsed with distilled water for 4-5 times and air-dried. Glacial acetic acid (10%) in Milli-Q water was added to extract the crystal violet stain. The absorbance was then measured at 595 nm using a microplate reader (Tecan Infinite® 200 Pro, Switzerland).

### EdU Incorporation Assay

EdU incorporation assay was performed using an EdU proliferative kit (iFluor 488) according to manufacturer's instructions. Briefly, 2.5×10⁴ HaCaT cells per well were seeded in a 96-well plate. After overnight incubation, cells were incubated with EdU (10 μM) for 4 h with bleogen pB1 or EGF in serum-free medium. Cells were then fixed, permeabilized, and incubated in a reaction mix containing iFluor 488 for 30 min. The nuclei were stained using DAPI. The plates were visualized using a fluorescence microscope.

### Cell Migration Assay

Cell migration was monitored using the scratch assay. Briefly, HaCaT cells were seeded into 2 well silicone inserts with a defined gap of 500 μm (ibidi, Germany). After overnight incubation, inserts were removed and incubated with bleogen pB1 or EGF in DMEM medium containing 0.1% FBS. After incubation for 8 h, the wells were photographed using inverted phase-contrast microscope. Images were analyzed for the percentage of cell-free area using the Wimasis image analysis platform.

### Endothelial Tube Formation Assay

Endothelial tube formation assay was performed using a μ-slide (ibidi, Germany). Briefly, 10 μL of gel matrix (BD Matrigel™ Basement Membrane Matrix) was casted onto each μ-slide well and left to solidify. HUVEC-CS cells were seeded per well at the density of 2,500 cells/well together with bleogen pB1 or EGF. After incubation for 2 h, the wells were photographed using inverted phase-contrast microscope. Images were analyzed for the numbering of branching points using the Wimasis image analysis platform.

### Pull-Down Assay

Pull-down assay was performed using NeutrAvidin UltraLink Resin (Thermo Fisher Scientific, US). Briefly, the resin was washed with and phosphate buffered saline (PBS) three times and incubated with biotin-pB1/biotin-[K29k]pB1/biotin-[Y25y]pB1, or biotin (negative control), or biotin-aB1 (negative control), or biotin-EGF (positive control) at room temperature with rotation for 2 h. 2% bovine serum albumin (BSA) in PBS was added to both tubes and incubated at room temperature with gentle end-over-end mixing for another 2 h. 600 μg of HaCaT cell lysate was added to each tube and allowed to incubate overnight at 4° C. with rotation. After incubation, the resin was transferred to Pierce® spin columns and washed 10 times with PBS. A 6× loading dye with 3-mercaptoethanol was added to the resin and heated for 10 min at 85° C. The resultant mixture was centrifuged at 200 g for one min and resolved using 8% Sodium dodecyl sulfate polyacrylamide gel electrophoresis (SDS-PAGE) at 100 V constant for 120 min. Blot transfer was performed on a polyvinylidiene difluoride (PVDF) membrane (GE healthcare, Sweden) at 250 mA for 120 min on ice. The blot was blocked with 5% BSA tris-buffered saline and tween 20 (TBST) before incubating overnight at 4° C. with anti-EGFR anti-rabbit antibody (1:1000 in 5% BSA TBST; Cell signaling, US). After incubation overnight, the membrane was washed with TBST at room temperature for three times at 10 min each. The blot was then incubated with secondary anti-rabbit horseradish peroxidase (HRP) (Cell Signaling, US) (1:5000 in 5% BSA TBST) and was incubated for another 1 h at room temperature. The blot was washed five times 10 min each with TBST at room temperature before the addition of chemiluminescence substrate (Advansta, US) and exposure on X-ray film (Fujifilm, Japan).

### Immunoprecipitation

Immunoprecipitation was performed using EGFR (D38B1) rabbit monoclonal antibodies conjugated to magnetic beads (Cell Signaling, US). Briefly, the magnetic beads were washed with PBS three times and 50 μg HaCaT cell lysate was added to each tube and allowed to incubate overnight at 4° C. with rotation. After incubation, the magnetic beads were washed five times with PBS. A 6× loading dye with β-mercaptoethanol was added to the resin and heated for 10 min at 85° C. The resultant mixture was centrifuged at 14000 g×1 min and resolved using 10% SDS-PAGE at a 100 V constant for 120 min. Blot transfer was performed on a PVDF membrane (GE healthcare, Sweden) at 250 mA for 120 min on ice. The blot was blocked with 5% BSA TBST before incubating overnight at 4° C. with anti-phosphotyrosine-HRP (R&D systems, US) or anti-EGFR anti-rabbit antibody (1:1000 in 5% BSA TBST; Cell Signaling, US). After incubation, the membrane was washed with TBST at room temperature for three times at 10 min each. The blot was then incubated with secondary anti-rabbit horseradish peroxidase (HRP) (1:5000 in 5% BSA TBST) and was incubated for another 1 h at room temperature. The blot was washed five times 10 min each with TBST at room temperature before the addition of chemiluminescence substrate (Advansta, US) and exposure on X-ray film (Fujifilm, Japan).

### Western Blot Analyses

Blot transfer was performed onto a PVDF membrane (GE healthcare, Sweden) at 250 mA for 120 min on ice. The blot was blocked with 5% BSA TBST before incubating overnight at 4° C. with anti-P-MEK1/2 rabbit antibody (1:2000 in 5% BSA TBST; Cell Signaling, US), anti-MEK1/2 rabbit antibody (1:2000 in 5% BSA TBST; Cell Signaling, US), anti-P-ERK1/2 rabbit antibody (1:2000 in 5% BSA TBST; Cell Signaling, US), anti-ERK1/2 rabbit antibody (1:2000 in 5% BSA TBST; Cell Signaling, US), and anti-β-actin mouse antibody (1:10000 in 5% BSA TBST; Merck Millipore, US). After incubation, the membrane was washed with TBST at room temperature three times for 10 min each. The blot was then incubated with secondary anti-mouse or anti-rabbit horseradish peroxidase (HRP) (1:5000 in 5% BSA TBST; Cell Signaling, US) and was incubated for another 1 h at room temperature. The blot was washed five times 10 min each with TBST at room temperature before addition of chemiluminescence substrate (Advansta, US) and exposure on X-ray film (Fujifilm, Japan).

### TR-FRET EGFR Ligand Binding Assay

Competitive displacement assay was performed using EGF-EGFR LANCE Ultra TR-FRET Binding Kit as per the manufacturer's instructions (PerkinElmer, US). Streptavidin is conjugated with LANCE Europium chelate which binds to biotin-EGF, whilst EGFR-Fc interacts with anti-human IgG that is labeled with ULight™ dye. Briefly, different concentrations of bleogen pB1 or its Ala- or D-amino acid analogs or EGF were mixed with the working solution and incubated at room temperature for 2 h. TR-FRET ratio was measured using a microplate reader in dual emission mode (Excitation: 340 nm, emission: 665 nm and 615 nm) (Cytation 1, US). The results were presented as the relative binding percentage of biotin-EGF. aB1 with an amino acid sequence of NCESGTSCIPGAQHNCCSGVCVPIVTIFYGVCY (SEQ ID NO:11) or roseltide rT7 with an amino acid sequence of CVSSGIVDACSECCEPDKCIIMLPTWPPRYVCSV (SEQ ID NO:17) were used as negative control. EGF (SEQ ID NO:10) was used as positive control.

### Luciferase Reporter Assay

Stable HaCaT cells expressing SRE luciferase reporter were tested for bioluminescence response to treatment with bleogen pB1 and EGF as positive control. Briefly, the cells were cultured in a white 96-well plates to achieve 90% confluency. The cells were washed with serum-free medium, starved overnight in serum-free DMEM medium and treated with bleogen pB1 and EGF for 6 h. The luciferase assay was performed using the ONE-Glo EX Luciferase Assay System (Promega, US) as per the manufacturer's instructions. Luminescence was measured using a microplate reader (Tecan Infinite® 200 Pro, Switzerland).

### Gene Expression Analysis

Total RNA was extracted from HaCaT and primary human keratinocytes using PureLink™ RNA mini kit (Thermo Fisher Scientific, US). First-strand cDNA was synthesised from 600 ng of total RNA using SuperScript™ II Reverse Transcriptase and Oligo(dT)12-18 (Thermo Fisher Scientific, US) according to the manufacturer's instructions. Quantitative PCR (qPCR) was performed with iTaq Universal SYBR Green Supermix (Bio-Rad, US) on a CFX96 Touch Real-Time PCR Detection System (Bio-Rad, US) for 40 cycles. PCR reaction (20 μL): 3 μL cDNA, 1 μL primer mix (10 μM), 6 μL DEPC-treated water and 10 μL mastermix. The pre-designed primer pairs (Origene, US) used in the qPCR reactions are as following: c-fos (NM_005252) were 5′-GCC TCT CTT ACT ACC ACT CAC C-3′ (forward; SEQ ID NO:18) and 5′-AGA TGG CAG TGA CCG TGG GAA T-3′ (reverse; SEQ ID NO:19); c-Jun (NM_002228) were 5′-CCT TGA AAG CTC AGA ACT CGG AG-3′ (forward; SEQ ID NO:20) and 5′-TGC TGC GTT AGC ATG AGT TGG C-3′ (reverse; SEQ ID NO:21); reference gene GAPDH (NM_001256799) were 5′-GTC TCC TCT GAC TTC AAC AGC G-3′ (forward; SEQ ID NO:22) and 5′-ACC ACC CTG TTG CTG TAG CCA A-3′ (reverse; SEQ ID NO:23). GAPDH was used as a housekeeping gene for normalization. Fold changes of gene expressions with c-fos and c-Jun were calculated using the 2^{-ΔΔCT} method.

### Full-Thickness Splinted Excisional Wounding of Mice

C57BL/6 mice were obtained from Vital Rital Laboratories (Beijing, China). Mice were housed in plastic cages at 23±1° C. with a 12-h light/dark cycle and free access to water and food. All experiments were approved and performed in accordance with the institutional guidelines of the Experimental Animal Center of the Chinese Academy of Medical Science (Beijing, China).

Full-thickness 5-mm splinted excisional wounding of mice was performed. Briefly, mice were anesthetized using an intraperitoneal injection of sodium pentobarbital (50 mg kg⁻¹). The hair of the back was shaved with an electric clipper followed by a depilatory cream. Using a 5-mm-diameter sterile biopsy punch, symmetrical full-thickness excisional wounds were created. Splints were placed around the wound using adhesive glue and secured with four interrupted sutures. The wounds and splints were covered with Tegaderm (3M, US). At indicated days within the 14 days post-injury course, wound diameters were measured. The size of wound was calculated from the average of two diameter measurements along the x and y axis.

### Treatment Regimen for Full-Thickness Splinted Excisional Wound Healing in C57BL/6 Mice

Full-thickness 5 mm splinted excisional wounding of mice was performed on C57BL/6 mice as described above. Post-injury, saline (vehicle control; n=30 wounds; 15 mice), EGF (1 nmol/wound, positive control; n=10 wounds; 5 mice), and bleogen pB1 (10 nmol/wound; n=10 wounds; 5 mice) were made to the wounds for three consecutive days.

### Treatment Regimen for Full-Thickness Splinted Excisional Wound Healing in Streptozotocin-Induced Diabetic C57BL/6 Mice

Diabetes was induced into C57BL/6 mice by two intraperitoneal injection (80 mg/kg) of STZ at day 1 and 5. Starting from day 8, blood glucose was monitored regularly for two weeks. Mice with blood glucose level above 11.1 mmol/L were considered as diabetic and subjected to full-thickness 5 mm splinted excisional wounding as described above (Total 24 mice, two wound per mice). Post-injury, saline (vehicle control; n=12 wounds), bleogen pB1 (1 nmol/wound; n=12 wounds), [K29k]pB1 (1 nmol/wound; n=12 wounds), and EGF (1 nmol/wound; n=12 wounds) were topically administered to the wounds for five consecutive days. Blood glucose was also monitored at day 14 post-injury to ensure the mice are diabetic.

### Newborn Mice Model for Incisor Eruption

ICR mice were obtained from Vital Rital Laboratories (Beijing, China). Mice were housed in plastic cages at 23±1° C. with a 12-h light/dark cycle and free access to water and food. All experiments were approved and performed in accordance with the institutional guidelines of the Experimental Animal Center of the Chinese Academy of Medical Science (Beijing, China). Subcutaneous injections were made for five consecutive days in the nape of the neck of the newborn mice starting from the day of birth (day 0). A total of 30 newborn mice were randomly separated into three test groups as follows: PBS, EGF (3 mg/kg), and bleogen pB1 (3 mg/kg). Incisor eruption was recorded daily by visual inspection. Incisor eruption was defined as the time at which a given tooth first pierced the oral epithelium.

### Peptide Stability Assay

### Heat Stability

0.1 M of purified bleogen pB1, [K29k]pB1, S-alkylated pB1 (iodoacetamido-), EGF, PBS (control) were incubated at 100° C. Samples were collected at various time points (0, 30, 60, and 120 min).

### Acid Stability

0.1 M of purified bleogen pB1, [K29k]pB1, S-alkylated pB1 (iodoacetamido-), and EGF was dissolved in 0.2 M HCl and incubated at 37° C. Samples were collected at various time points.

### Pepsin Stability

0.1 M of purified bleogen pB1, [K29k]pB1, S-alkylated pB1 (iodoacetamido-), and EGF was dissolved in 0.2 M HCl and incubated with pepsin (Roche Applied Science, US) in a 50:1 (w/v) ratio at 37° C. Samples were collected at various time points.

### Pronase Stability

0.1 M of purified bleogen pB1, [K29k]pB1, S-alkylated pB1 (iodoacetamido-), and EGF was dissolved in PBS and incubated with pronase (0.2 mg/mL; Roche Applied Science, US) at 37° C. Samples were collected at various time points.

### Neutrophil Elastase Stability

0.1 M of purified bleogen pB1, [K29k]pB1, S-alkylated pB1 (iodoacetamido-), and EGF was dissolved in PBS and incubated with human neutrophil elastase (0.05 mg/mL; Molecular Innovations, US) at 37° C. Samples were collected at various time points.

### Trypsin Stability

0.1 M of purified bleogen pB1, [K29k]pB1, S-alkylated pB1 (iodoacetamido-), and EGF was dissolved in PBS and incubated with trypsin (0.2 mg/mL; Sigma Aldrich, US) at 37° C. Samples were collected at various time points.

### Human Serum Stability

0.1 M of purified bleogen pB1, [K29k]pB1, S-alkylated pB1 (iodoacetamido-), and EGF were prepared in 25% human serum in DMEM medium without phenol red. The test samples were incubated at 37° C. Samples were collected at various time points. The collected samples were subjected to protein precipitation with 100% ethanol and centrifugation at 180,000 g for 5 min at 4° C. The supernatant was collected for analysis.

### Analysis for Stability Assays

All collected samples from various stability assays were analyzed by RP-HPLC with a linear gradient of mobile phase A (0.05% TFA/H₂O) and mobile phase B (0.05% TFA/ACN) on aeris peptide XB-C18 column (Phenomenex, US). The resulting peaks were collected and identified by MALDI-TOF MS. The results were expressed as percentage of initial concentration using the peak area of the HPLC profile.

### Statistical Analyses

Statistical comparisons were performed using GraphPad Version 8.2.1 (US). Data were analyzed using one-way analysis of variance (ANOVA) followed by Newman-Keuls post hoc tests. Data were expressed as mean±S.D. and p<0.05 was considered to be statistically significant.

### Example 1: Motif Search and Molecular Docking of Bleogen pB1 as an EGFR Agonist to Determine the “Hot Spot” for Structure-Activity Relationship Studies

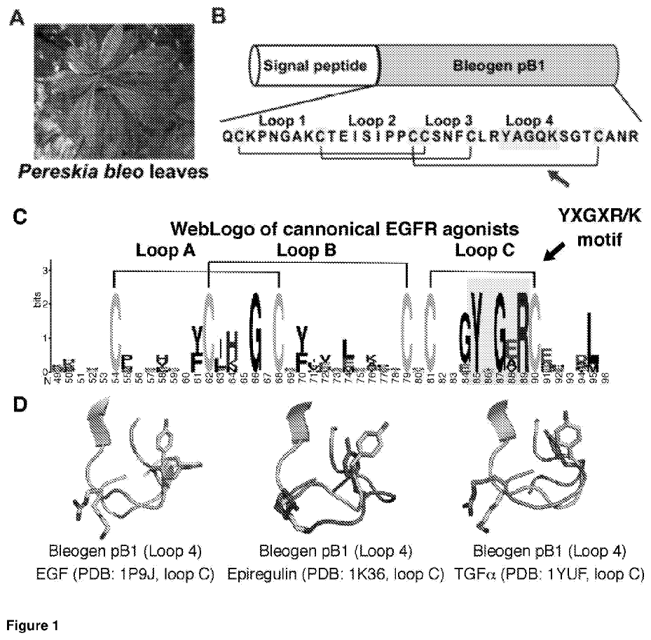

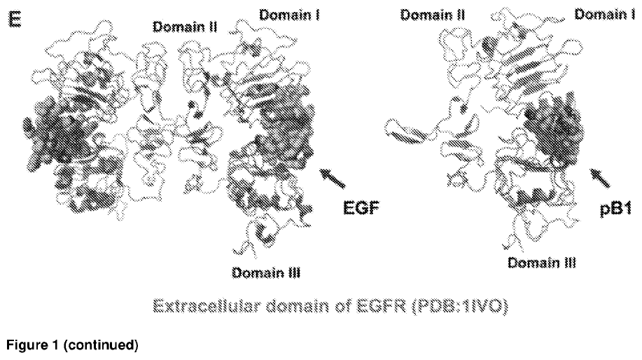

The seven related mammalian EGFR agonists, including EGF, TGF-α, Hb-EGF, betacellulin, amphiregulin, epiregulin, and epigen, share four conserved non-cysteine residues, three of which are located in loop C (Cys V-VI), forming a specific YXGXR motif (X, any amino acid) (FIG. 1C). The loop 4 of bleogen pB1 also contains an YXGXK motif, similar to the loop C of EGFR agonists in sequence and structure (FIG. 1D). In silico modeling of bleogen pB1 and EGFR (PDB entry 1IVO, chain A) using protein-protein docking server ClusPro Version 2.0 showed that bleogen pB1 loop 4 could bind to EGFR at the same site as EGF loop C, suggesting a common “hot spot” for binding to EGFR (FIG. 1E) (Ogiso, supra). Importantly, the discovery of this common “hot spot” shared by pB1 and EGFR agonists provided grounds for our subsequent structure-activity relationship studies.

### Example 2: Synthesis and Characterization of Bleogen pB1

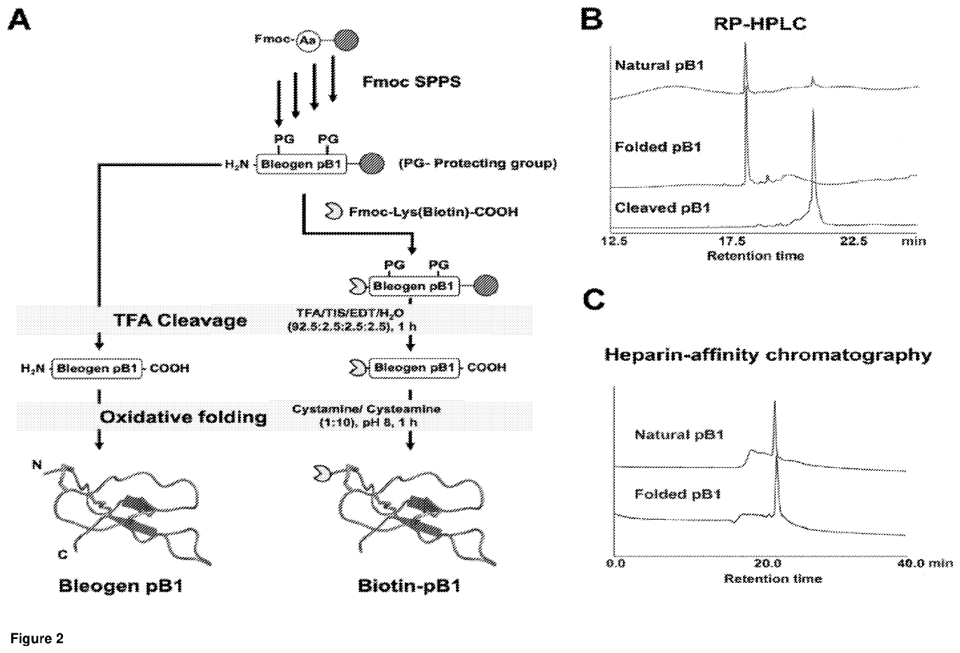

For both in vitro and in vivo assays of EGF-like activities, the natural bleogen pB1 isolated from the Pereskia plant was used. The natural bleogen pB1 was isolated from the aqueous leaf extracts of Pereskia bleo, using C-18 reversed-phase high performance liquid chromatography (RP-HPLC) (Loo et al. (2017) Bleogens: cactus-derived anti-Candida cysteine-rich peptides with three different precursor arrangements. Front. Plant Sci. 8:2162). To prepare synthetic bleogen pB1, a stepwise solid-phase method and Fmoc chemistry was used (FIG. 2A). After removing the protecting groups and cleaving the unprotected peptide from the resin support by trifluoroacetic acid (TFA), the crude pB1 product was oxidatively folded using a combination of redox reagents consisting of cysteamine and cystamine in 10:1 molar ratio in 0.1 M ammonium bicarbonate, pH 8 for 1 h to give 70% yield of bleogen pB1. The purified synthetic and natural bleogen pB1 were indistinguishable as demonstrated by RP-HPLC, heparin-affinity chromatography, and 2D-Nuclear magnetic resonance (NMR) (FIG. 2 B, C).

To equip bleogen pB1 with a chemical affinity probe for target identification (FIG. 2A), total synthesis was used again by coupling Fmoc-Lys(biotin) to the N-terminus of the protected pB1 peptide still attached to resin supports to give the biotinylated-pB1 (biotin-pB1). These total syntheses provided unambiguous site-specific labeling of bleogen pB1 at its N-terminus to retain the integrity of their side chain functional groups.

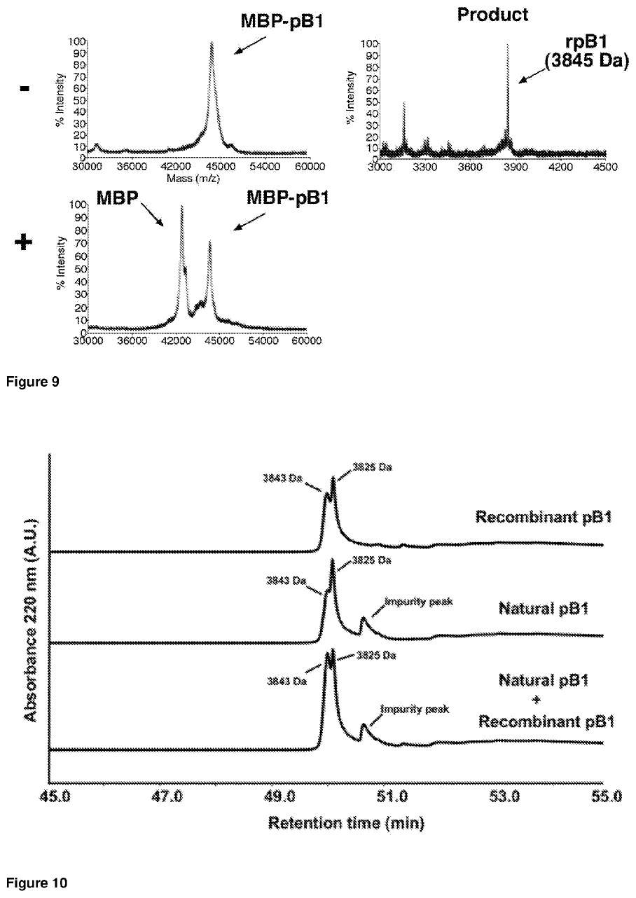

Additionally, bleogen pB1 was also produced recombinantly as MBP (maltose binding protein) fusion protein in E. coli using LB medium using IPTG-induced expression (0.4 mM for 4 h at 37° C.; 0.1 mM for 20 h at 15° C.; data not shown). For purification, the expressed fusion protein was isolated by affinity chromatography using a maltose resin and elution with 10 mM maltose in PBS. The purification yield of MBP-pB1 was around 10-15 mg per L of LB broth. The MBP tag was removed by treatment with enterokinase, as demonstrated by mass spectrometry analysis (FIG. 9). FIG. 10 shows that both natural and recombinant bleogen pB1 co-eluted at the same retention time using RP-HPLC.

### Example 3: Bleogen pB1 Displays EGF-Like Biological Activities

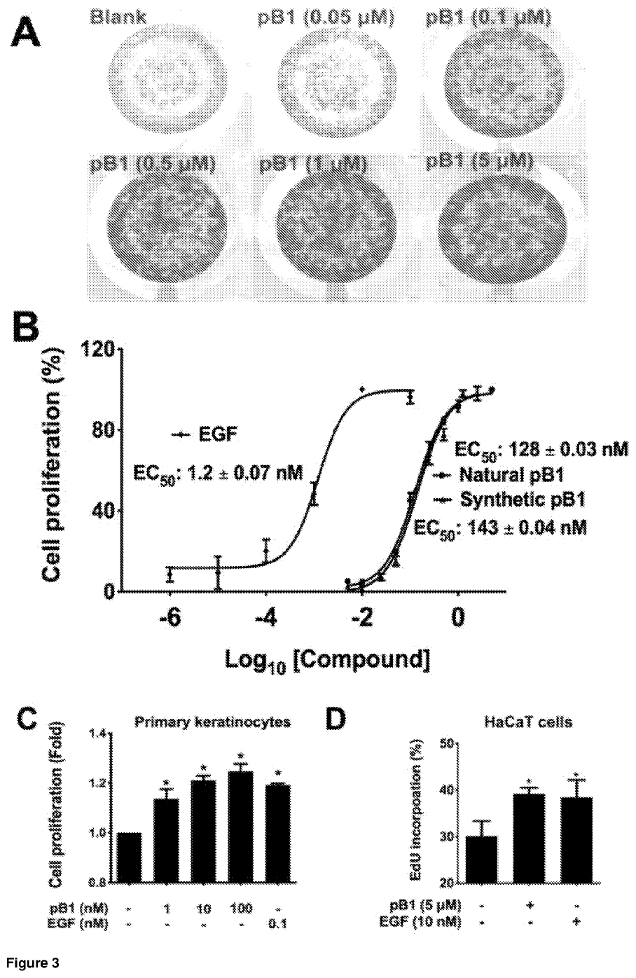

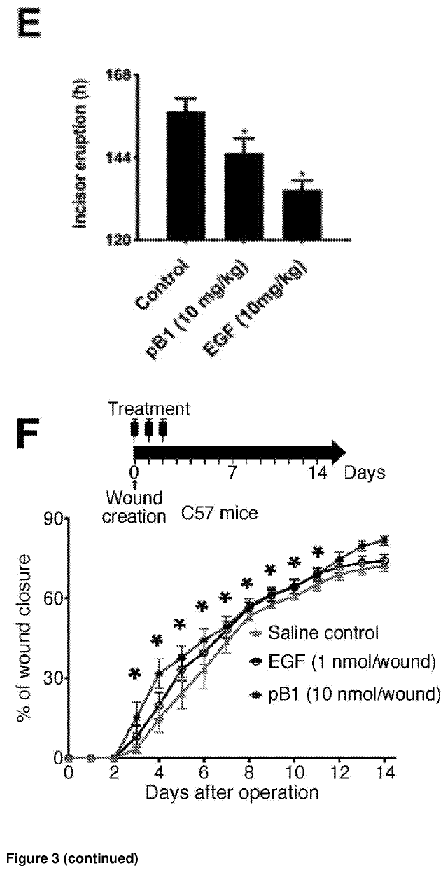

To determine whether bleogen pB1 is an EGF-like mitogen, its biological effects on HaCaT keratinocyte proliferation were examined using EGF as a positive control. It was shown that bleogen pB1 and EGF promotes HaCaT cell proliferation with an EC50 of 130 nM and 1.2 nM, respectively (FIG. 3A, B). The 100-fold difference in mitogenic potency between EGF and pB1 suggests that bleogen pB1 belongs to the family of low-affinity EGFR agonists such as epigen and amphiregulin. Both native and synthetic bleogen pB1 showed identical proliferative activities on HaCaT cells, confirming that the observed proliferative effects were not due to contaminants from the plant extracts (FIG. 3B). As a negative control, the 3417-Da-peptide aB1 isolated from Achyranthes bidentata, a 6CHLP belonging to the same CRP family as bleogen pB1 with similar cysteine motif and disulfide connectivity, was not active up to 10 μM in this assay. It was also shown that bleogen pB1 promotes the proliferation of primary human keratinocytes (FIG. 3C), and enhances DNA synthesis of HaCaT cells using 5-ethynyl-2′-deoxyuridine (EdU) incorporation assay (FIG. 3D). In addition, both EGF and bleogen pB1 accelerate HaCaT cell migration and endothelial cell tube formation (data not shown). A hallmark EGF assay that led to its discovery is the incisor eruption in newborn mice. It was shown that subcutaneous injection of bleogen pB1 (3 mg/kg) or EGF (3 mg/kg) for five consecutive days, accelerated the incisor eruption in newborn mice, from 153 h (saline control) to 125 h and 100 h, respectively (FIG. 3E). It was also shown that bleogen pB1 displays in vivo wound healing. Treatment with bleogen pB1 (10 nmol/wound) or EGF (1 nmol/wound) for three consecutive days accelerated wound healing from day 3 to day 11 using the full-thickness excision wound model in C57 mice (FIG. 3F) (Wang et al. (2013), Nat. Protoc. 8(2):302). Taken together, these six different in vitro and in vivo assays strongly support that bleogen pB1 is a mitogen and exerts EGF-like biological activities.

### Example 4: Bleogen pB1 Interacts with EGFR

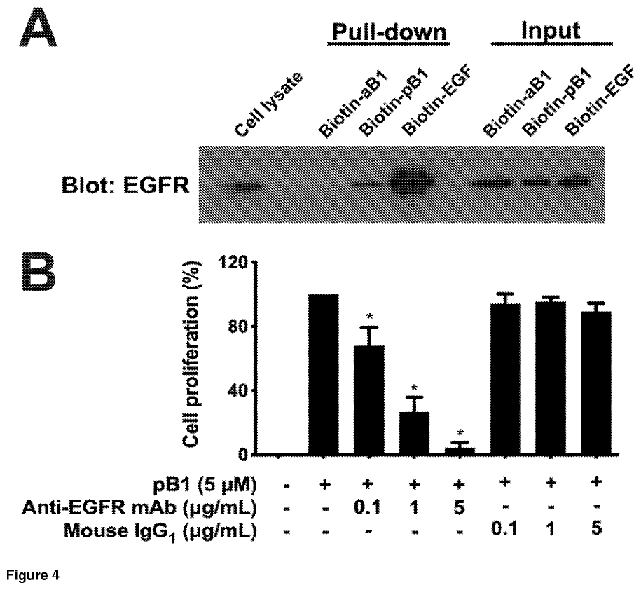

To show that the mitogenic activity of bleogen pB1 is a result of its interaction with EGFR, pull-down and neutralizing antibody assays were used to examine their interactions. Biotin-pB1 was able to pull-down EGFR, suggesting their putative ligand-receptor interaction (FIG. 4A). Co-incubation of an EGFR neutralizing antibody (clone LA1) blocked bleogen pB1-induced HaCaT cell proliferation (FIG. 4B). These results strongly supported that the interaction between bleogen pB1 and EGFR is specific, and the proliferative effect is EGFR-dependent.

### Example 5: Proliferative Effect of Bleogen pB1 is Associated with the EGFR/MEK/ERK Signaling Pathway

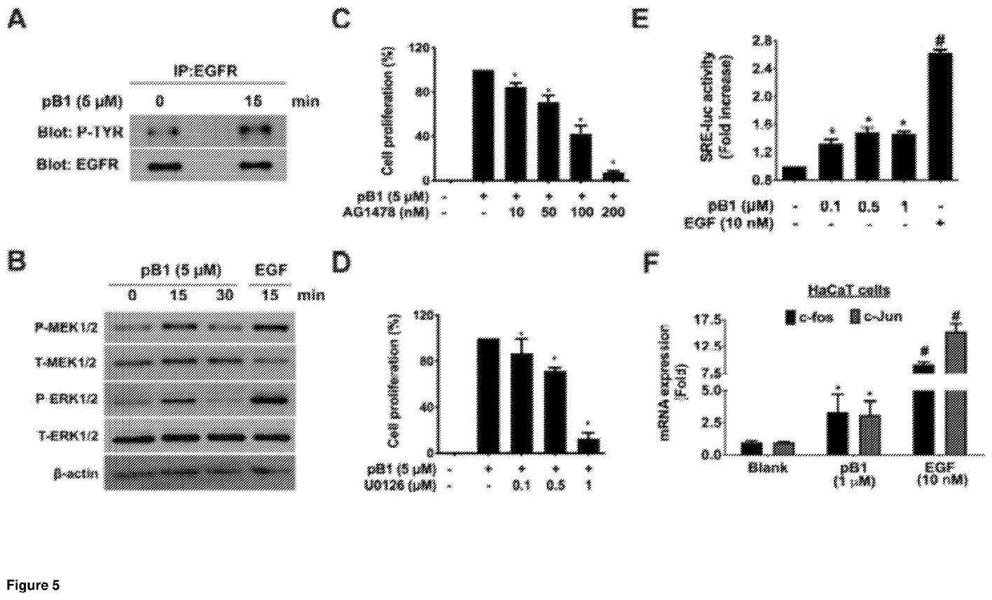

EGF activates the EGFR/MEK/ERK signaling pathway which has an important role in regulating cell proliferation. It was explored whether bleogen pB1 would undergo a similar EGF signaling pathway to induce keratinocyte proliferation. We found that bleogen pB1 induces the phosphorylation of EGFR, MEK1/2, and ERK1/2 (FIG. 5A,B). Furthermore, co-incubation with a small molecule EGFR tyrosine kinase inhibitor (AG1478) or MEK inhibitor (U0126) substantially inhibited the proliferative effects of bleogen pB1 in HaCaT cells (FIG. 5C,D). Collectively, these results suggest that bleogen pB1 binds to EGFR and activates the EGFR/MEK/ERK signaling pathway to trigger HaCaT cell proliferation.

EGF is known to activate transcription factors that bind to serum response element (SRE) and initiate transcription of immediate early genes involved in the regulation of EGFR-mediated cell proliferation, such as c-fos and c-Jun (Lee et al. (2018) Sci. Rep. 8(1):162). It could be shown that bleogen pB1, dose-dependently, increases the luciferase activity, similar to EGF in a stably-expressed SRE-luciferase reporter HaCaT cell line (FIG. 5E), indicating that bleogen pB1 stimulates SRE-mediated gene transcription. Also examined were the effects of EGF- and bleogen pB1-induced transcriptional response on the expressions of EGF-associated immediate early genes using qPCR (Brankatschk et al. (2012), Sci. Signal. 5(215):ra21-ra21). It was confirmed that both bleogen pB1 and EGF significantly upregulate the mRNA expressions of c-fos and c-Jun in HaCaT cells (FIG. 5F). Taken together, this series of experiments support the similarity of EGF- and pB1-induced signaling pathways and transcriptional response.

### Example 6: Positional-Scanning of the Loop 4 Containing YAGQK Motif in Bleogen pB1 Using Ala- and D-Amino Acid

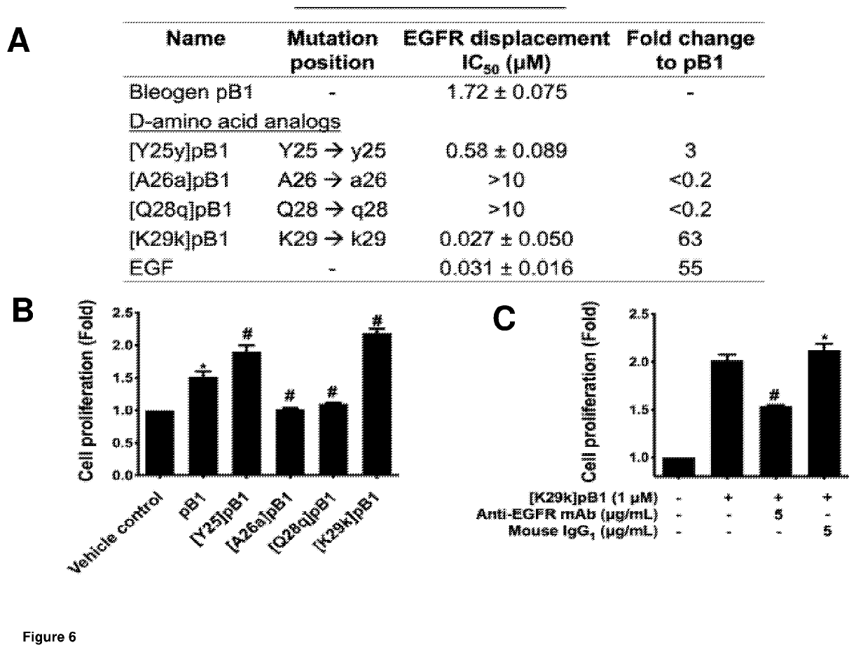

It was hypothesized that the YXGXK/R motif (X, any amino acid; SEQ ID NO:3) in loop 4 of bleogen pB1 and the loop C of canonical EGFR agonists could be a common “hot spot” for EGFR interaction. Previously, it was demonstrated that mutations of Y38 and R42 in TGF-α YXGXR motif loop C using Ala- or D-amino-acid scan resulted in a substantial decrease in both EGFR affinity and EGF-like mitogenic potential (Tam and Tam et al., supra). Accordingly, a focused Ala- and D-amino acid library of the corresponding “hot spot” of bleogen pB1, YAGQK loop 4 was chemically synthesized. This included the Ala-substituted series of [Y25A]pB1, [G27A]pB1, [Q28A]pB1, and [K29A]pB1 and the D-amino acid-substituted series of [Y25y]pB1, [A26a]pB1, [Q28q]pB1, and [K29k]pB1. Each peptide was compared with bleogen pB1 in two different studies, to test its affinity to EGFR using time-resolved fluorescence energy transfer (TR-FRET)-based competitive displacement and its mitogenic potential using HaCaT cell proliferation assay. The results showed that bleogen pB1 displaces biotin-EGF with an IC50 of 1720±0.075 nM, whereas EGF has an IC50 of 31±0.016 nM (FIG. 6A). All Ala-analogs showed a decrease in EGF-like biological activities using both EGFR displacement and cell proliferation assays (data not shown). In contrast, two D-analogs, [K29k]pB1 and [Y25y]pB1, displayed higher affinity than bleogen pB1, with an IC50 of 27±0.050 nM and 580±0.089 nM, respectively. Compared to bleogen pB1, [K29k]pB1 is approximately 60-fold more potent (FIG. 6B). It was shown that biotin-[K29k]pB1 and biotin-[Y25y]pB1, chemically synthesized as described previously for biotin-pB1, interact with EGFR using a pull-down assay (data not shown). To confirm this data, it was shown that [K29k]pB1 and [Y25y]pB1 are more mitogenic than bleogen pB1, and their mitogenic effects can be inhibited by EGFR neutralizing antibody (clone LA1), indicating that their activity is EGFR-dependent (FIG. 6C).

### Example 7: Bleogen pB1 Accelerates Wound Healing in Excisional Wound Model Using Streptozotocin-Induced Diabetic Mice

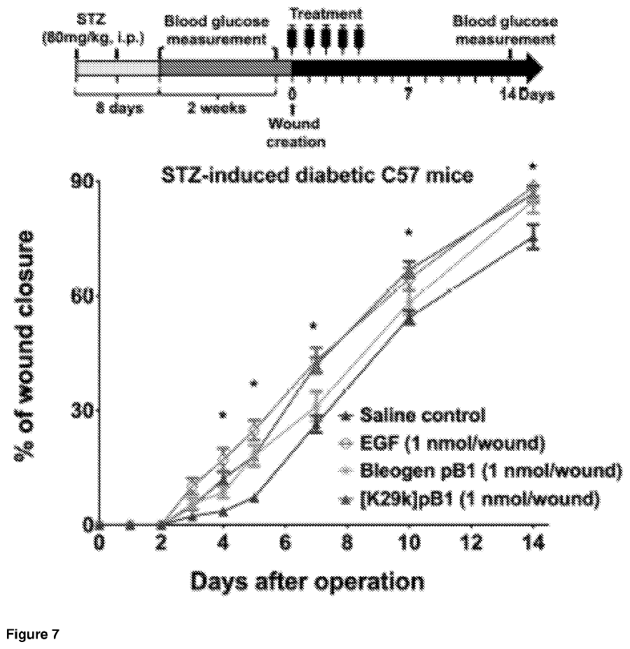

It was explored whether the enhanced mitogenic effects of [K29k]pB1 observed the in vitro assays would also accelerate wound healing in streptozotocin (STZ)-induced diabetic mice. Diabetes was induced in C57 mice by two intraperitoneal injections (80 mg/kg) of STZ on day 1 and 5. Starting from day 8, blood glucose was monitored regularly for two weeks. Mice with blood glucose levels above 11.1 mmol/L were considered diabetic and subjected to the splinted excisional wound healing model (Huang et al. (2018), FASEB J. 33(1):953-964). It was shown that wound closure for STZ-induced diabetic mice is significantly delayed as compared to non-diabetic mice. It was also shown that wounds topically treated with EGF (1 nmol/wound), bleogen pB1 (1 nmol/wound) or [K29k]pB1 (1 nmol/wound) for five consecutive days, healed at a significantly faster rate starting from day 4 post-injury compared to the vehicle control group, macroscopically (FIG. 7). Notably, [K29k]pB1 promoted wound closure at a similar rate as EGF, and much faster than bleogen pB1. These in vivo results are in agreement with the in vitro assays on EGFR affinity and mitogenicity, suggesting that [K29k]pB1 is of equal potency as EGF.

### Example 8: Bleogen pB1 and [K29k]pB1 are Hyperstable Against Proteolytic Degradation

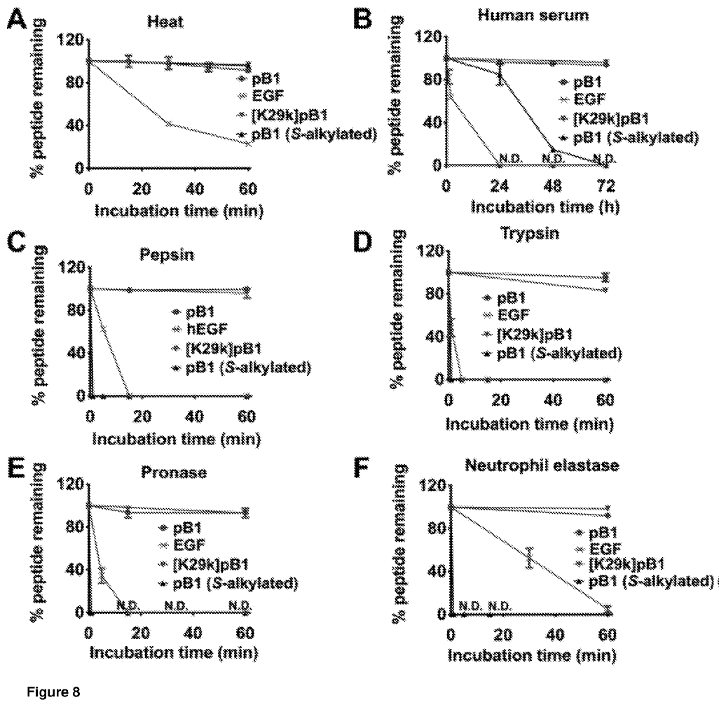

To determine whether bleogen pB1 and [K29k]pB1, which are structurally more compact than EGF, are more proteolytic stable and have resistance against heat and acid treatments, all three tested peptides, bleogen pB1, [K29k]pB1, and EGF, are relatively stable to acid with a calculated t1/2>500 min (data not shown). It was however observed that bleogen pB1 and [K29k]pB1 were remarkably more stable than EGF in heat treatment with a calculated t1/2>500 min (FIG. 8A). More importantly, they were also subjected to a panel of diverse proteases to test their proteolytic stability. These included pepsin, human serum, trypsin, pronase, and human neutrophil elastase (FIG. 8B-F). Bleogen pB1 and [K29k]pB1 showed stark contrast when compared to EGF in their susceptibility to our panel of proteases. The t1/2 of bleogen pB1 and [K29k]pB1 were calculated to be >500 min in the panel of proteases and >800 h in human serum. In contrast, the t1/2 of EGF ranged from 0.9 to 23.5 min in the presence of the panel proteases and 3.1 h in human serum. EGF was found to be at least 100-fold less stable under proteolytic conditions as compared to bleogen pB1 and [K29k]pB1. The control peptide, the S-alkylated bleogen pB1 with all its disulfide bridges reduced and S-alkylated by iodoacetamide, was degraded by proteases within a couple of minutes, suggesting that the structural integrity of bleogen pB1 and [K29k]pB1 contributes significantly to their proteolytic stability.

1. A recombinant peptide having EGFR-binding activity, said peptide comprising or consisting of
(i) the amino acid sequence as set forth in SEQ ID NO:1;
(ii) an amino acid sequence that shares at least 60%, optionally at least 70% sequence identity with the amino acid sequence set forth in SEQ ID NO:1 over its entire length;
(iii) an amino acid sequence that shares at least 80% sequence homology with the amino acid sequence set forth in SEQ ID NO:1 over its entire length; or
(iv) a fragment of any one of (i)-(iii), wherein said fragment comprises the amino acids corresponding to amino acid residues 2-33 of SEQ ID NO:1.

2. The recombinant peptide of claim 1, wherein the recombinant peptide comprises
(i) the amino acid sequence motif C(X)nC(X)mCC(X)oC(X)pC, wherein X can be any amino acid with the exception of C, n is an integer from 4 to 8, m is an integer from 5 to 9, o is an integer from 1 to 5, and p is an integer from 8 to 12; and/or
(ii) the amino acid sequence motif YXGXK/R (SEQ ID NO:3), wherein X can be any amino acid with the exception of C.

3. The recombinant peptide of claim 1, wherein said recombinant peptide comprises the amino acid sequence motif C(X)ₙC(X)ₘCC(X)ₒC(X)ₚC, wherein X can be any amino acid with the exception of C, n is an integer from 4 to 8, m is an integer from 5 to 9, o is an integer from 1 to 5, and p is an integer from 8 to 12, wherein (X)ₚ comprises the amino acid sequence motif YXGXK/R (SEQ ID NO:3), wherein X can be any amino acid with the exception of C.

4. The recombinant peptide of claim 1, wherein the recombinant peptide comprises one or more D-amino acids.

5. The recombinant peptide of claim 4, wherein the recombinant peptide comprises one or more D-amino acids in
(i) the position corresponding to position 25 of SEQ ID NO:1; and/or
(ii) the position corresponding to position 29 of SEQ ID NO:1.

6. (canceled)

7. The recombinant peptide of claim 1, wherein said recombinant peptide has a positive net charge.

8. The recombinant peptide of claim 1, wherein said recombinant peptide comprises one, two or three disulfide bridges.

9. The recombinant peptide of claim 1, wherein said recombinant peptide has an at least 2-fold higher stability toward heat and/or protease compared to human EGF having the amino acid sequence set forth in SEQ ID NO:10.

10-14. (canceled)

15. A composition comprising a recombinant peptide of claim 1 and optionally a carrier and/or excipient.

16. The composition of claim 15, wherein the composition is a cosmetic, pharmaceutical or cosmeceutical composition.

17-20. (canceled)

21. A method for treating or preventing an EGF- or EGFR-related disease or disorder in a subject in need thereof comprising administering a therapeutically or prophylactically effective amount of a recombinant peptide of claim 1 to said subject.

22. The recombinant peptide of claim 1, wherein said recombinant peptide comprises or consists of
(i) an amino acid sequence that shares at least 80% sequence identity with the amino acid sequence set forth in SEQ ID NO:1 over its entire length; or
(ii) an amino acid sequence that shares at least 90% sequence homology with the amino acid sequence set forth in SEQ ID NO:1 over its entire length.

23. The recombinant peptide of claim 1, wherein said recombinant peptide comprises or consists of
(i) an amino acid sequence that shares at least 90% sequence identity with the amino acid sequence set forth in SEQ ID NO:1 over its entire length; or
(ii) an amino acid sequence that shares at least 95% sequence homology with the amino acid sequence set forth in SEQ ID NO:1 over its entire length.

24. The recombinant peptide of claim 1, wherein the recombinant peptide comprises
(i) the amino acid sequence motif C(X)6C(X)7CC(X)3C(X)10C (SEQ ID NO:2), wherein X can be any amino acid with the exception of C; and/or
(ii) the amino acid sequence motif YXGXK/R (SEQ ID NO:3), wherein X can be any amino acid with the exception of C.

25. The recombinant peptide of claim 1, wherein said recombinant peptide comprises the amino acid sequence motif C(X)6C(X)7CC(X)3CXXYXGXK/RXXXC (SEQ ID NO:4), wherein X can be any amino acid with the exception of C.

26. The recombinant peptide of claim 1, wherein said recombinant peptide comprises the amino acid sequence motif CK/RPXGXK/RCXXXXXPPCCXXXCXK/RYXGXK/RXGXCXXK/R (SEQ ID NO:5), wherein X can be any amino acid with the exception of C.

27. The recombinant peptide of claim 1, wherein said recombinant peptide comprises the amino acid motif CKPXGXKCXEXXXPPCCXXXCXRYXGXKXGXCXXR (SEQ ID NO:6), wherein X can be any amino acid with the exception of C.

28. The recombinant peptide of claim 1, wherein the recombinant peptide comprises
(i) one or more D-tyrosine in the position corresponding to position 25 of SEQ ID NO:1; and/or
(ii) one or more D-lysine in the position corresponding to position 29 of SEQ ID NO:1.

29. The recombinant peptide of claim 1, wherein the recombinant peptide comprises one, two or three disulfide bridges selected from disulfide bridges between C2 and C18, C9 and C22, and C17 and C33, using the positional numbering of SEQ ID NO:1.

30. The recombinant peptide of claim 1, wherein said recombinant peptide has an at least 5-fold higher stability toward heat and/or protease compared to human EGF having the amino acid sequence set forth in SEQ ID NO:10.
